(19) United States
(12) Patent Application Publication
(10) Pub. No.: US 2010/0075306 A1
(43) Pub. Date: Mar. 25, 2010

(54) METHOD FOR DIAGNOSIS OF AND FOLLOWING A BACTERIAL VAGINOSIS BY MOLECULAR QUANTIFICATION

(75) Inventors: Florence Bretelle, Marseille (FR); Florence Fenollar, Marseille (FR); Mireille Henry-Mary, Greasque (FR); Jean-Pierre Menard, Marseille (FR); Didier Raoult, Marseille (FR)

Correspondence Address: OLIFF & BERRIDGE, PLC, P.O. BOX 320850, ALEXANDRIA, VA, 22320-4850, US

(73) Assignee: UNIVERSITE DE LA MEDITERRANEE (AIX-MARSEILLE II), MARSEILLE (FR)

(21) Appl. No.: 12/312,711

(22) PCT Filed: Nov. 20, 2007
(86) PCT No.: PCT/FR2007/052368

§ 371 Date: Aug. 26, 2009

(30) Foreign Application Priority Data
Nov. 24, 2006 (FR) 06 10313

(51) Int. Cl. C12Q 1/68

(52) U.S. Cl. 435/6

(57) ABSTRACT

The present invention relates to a method for in vitro diagnosis and follow-up of vaginal bacterial flora status in relation to the presence of bacterial vaginosis, and for monitoring its treatment where applicable, wherein the presence of bacterial vaginosis or failure of on-going treatment is determined if the concentrations of specific sequences present in a single copy in the DNA of the bacteria Atopobium vaginae and Gardnerella vaginalis in the DNA extracted from a vaginal discharge specimen from a patient are such that at least one of the following two conditions a) and b) are met: a) the concentration Ca of said DNA fragment of Atopobium vaginae is greater than or equal to 10⁸ copies/mL, and b) the concentration Cg of said DNA fragment of Gardnerella vaginalis is greater than or equal to 10⁹ copies/mL.

The present invention relates to an in vitro method of diagnosis and follow-up of vaginal bacterial flora status relating to the presence of bacterial vaginosis (BV) or its outcome, where applicable, for following its therapeutic management.

### BACKGROUND

The current techniques for diagnosing BV, which is a common infection with harmful consequences for pregnancy, are based on unreliable criteria. Some bacteria have been described as being associated with this disease, but have never benefited from reliable molecular quantification and qualification.

For a long time, BV has been defined from the microbiological standpoint by near-disappearance of the normal vaginal flora composed mainly of lactobacilli, which have yielded to other bacteria, particularly Gardnerella vaginalis, Mobiluncus spp., and genital mycoplasmas [Spiegel C A, CMR 1991; Thorsen P, AJGO 1998]. BV brings many women into the doctor's office, and is particularly involved in susceptibility to sexually transmitted infections such as HIV, and in the case of pregnancy in prematurity and low-birth-weight babies. Its prevalence in women, including pregnant women, is between 8 and 23% [Guise J M, AJPM 2001] according to current research methods.

However, a review of the literature shows controversy as to the impact on the therapeutic management of this pathology. The early studies, which showed a reduction in the risk of premature labor when BV was treated, could not be confirmed [Morales H J, AJOG 1994; Hauth J C, NEJ M 1995; Guise J M, AJPM 2001; McDonald H, CDSR 2005; Varma R, EJOGRB 2006; Okun N, OG, 2005; Leitich H, AJOG 2003; Guerra B, EJOGRB 2006].

The initial objective of the present invention was to evaluate the impact of BV during pregnancy, and the efficacy of therapeutic management of BV during pregnancy. However, no objective diagnostic tools were available for doing so. A study of the literature reveals great confusion regarding the therapeutic management of BV, mainly due to the absence of rational tools for diagnosis and follow-up of this disease.

The two diagnostic tools that are available at the present time are the Nugent score and the Amsel criteria. The Nugent score is the method most frequently reported in the literature and is considered the gold standard by some, even though it is not routinely used in clinical microbiology laboratories because its implementation is cumbersome [Fredricks D N, NEJ M 2005; Thomason J L, AJOG 1992; Ison C A, STD 2002; Nugent R P, JCM 1991]. The Nugent score identifies BV by a semi-quantitative morphological analysis of the bacteria after Gram staining. Hence it is a subjective technique, whose reproducibility has been questioned [Sha B E, JCM 2005; Schwebke J R, OG 1996]. The clinical criteria of Amsel (vaginal pH over 4.5; grayish homogenous clinging discharge; nitrogen odor after addition of 10% KOH; presence of clue cells) represent the second diagnostic approach [Amsel R AJM 1983]. Like the Nugent score, it is cumbersome and not used in routine clinical practice.

One of the most harmful limitations of these diagnostic methods is their failure to identify certain microorganisms involved in BV. On the one hand, the mycoplasmas have no walls so cannot take a Gram stain and be scored by the Nugent method. On the other hand, knowledge from molecular biology has enabled new bacteria possibly involved in BV to be identified, but their demonstration by the two existing diagnostic methods is impossible. Atopobium vaginae is the main new bacterial species characterized. Its presence has been correlated with BV in some articles, but no reliable quantitative estimate of its position relative to other microorganisms has been made [Bradshaw C S, JID 2006, Rodriguez J M, IJSB 1999; Ferris M J, BMCID 2004; Ferris M J, JCM 2004; Verhelst R, BMCM 2004].

In the article recently published by Bradshaw et al. [Bradshaw C S, JID 2006], a relationship was described between detection of the bacteria A. vaginae and G. vaginalis and BV, but these results are insufficient for making a BV diagnosis and/or reliably tracking the outcome of BV. The data presented in this article enable these bacteria to be screened but not actually quantified. Additionally, this screening showed good sensitivity, as A. vaginae and G. vaginalis were detected in 96% and 99% respectively of BV patients. However, its specificity is poor, because A. vaginae was detected in 12% of patients with normal flora and G. vaginalis in 60%.

The authors then attempted a “semi-quantitative” approach by classifying the bacterial loads as low or high by comparison to mean CTs (cycle thresholds) of microorganism detection in the specimens analyzed. Thus, the authors estimated a median load of 4×10⁵ copies for G. vaginalis (median corresponding to 21 cycles) and 4×10⁶ copies for A. vaginae (median corresponding to 18 cycles). High loads of G. vaginalis (>4×10⁵) and A. vaginae (>4×10⁶) were significantly more present in BV patients than in patients with normal flora. However, these values have poor sensitivity, as A. vaginae and G. vaginalis were detected in only 49% and 71% of BV patients. See Bradshaw et al. at Table 1. Moreover, 16 patients (28%) with a relapse of BV after treatment had a G. vaginalis concentration below the given threshold. See Bradshaw et al. at Table 3. Forty patients (70%) with a relapse of BV also had an A. vaginae concentration below the threshold.

Hence, the “semi-quantitative” approach of the authors is applicable only as a tool for diagnosis and immediate follow-up of patients. The techniques used for obtaining these results were inadequate in several respects. First, the PCR techniques were not sensitive enough because the fragments amplified by the molecular targets were too long (16S ribosomal RNA 430 base pairs for A. vaginae and 291 base pairs for G. vaginalis). It has now been established that with a targeted sequence in such a long PCR reaction, the sensitivity is low. Moreover, the real-time PCR techniques use SybrGreen labeling of the amplification product for detection and quantification, which is a less specific method than those using labeled hydrolysis probes which need triple specificity (two primers plus the probe to amplify a fragment not exceeding 120 base pairs in size). The quantifications are probably done with a variable standard and not as a function of a stable, reproducible plasmid range that is comparable over time. Finally, they do not benefit from a quantitative control tool that enables specimen quality to be assessed: it looks only for the presence of human β-globin in the samples, without quantifying them. Hence it was very difficult to compare the samples with each other quantitatively, because the variation in the quantity of bacteria may be linked to a qualitative and quantitative variation in the vaginal secretions sampled.

### SUMMARY

Hence, for evaluation of the therapeutic management of BV, development of a reliable tool for BV diagnosis and qualitative and quantitative monitoring of the vaginal flora is indispensable.

The goal of the present invention is to provide a method for BV diagnosis and monitoring that is both more reliable, more precise, and easier to use routinely in clinical microbiology analysis laboratories.

For this purpose, the inventors studied vaginal specimens from 204 pregnant women, and looked for each microorganism implicated in BV, developing a real-time PCR method allowing the DNA of specific bacteria to be detected and the bacterial load to be determined by a plasmid range established by construction of a reference plasmid. This plasmid has the specific DNA fragments of said bacteria to be amplified and quantified and of the human albumin gene used to control the quality of the DNA specimen and of molecular amplification, as well as the richness of the biological specimen. The target microorganisms studied (Lactobacillus sp., G. vaginalis, Mobilincus curtisii, Mobilincus mulieris, Ureaplasma urealyticum, Mycoplasma hominis, A. vaginae, and Candida albicans) were those described as being possibly implicated in BV and/or prematurity. The results of quantifying the various microorganisms obtained by molecular biology were compared to the classification by the Nugent score.

According to the present invention, it has been demonstrated that the presence of A. vaginae starting at a certain threshold concentration may be very specifically and significantly associated with BV, and that this molecular detection makes the diagnosis easy and reliable.

The existence of a specific BV threshold concentration for the bacterium G. vaginalis has also been demonstrated according to the present invention.

Also, it has been demonstrated that the combination of the two bacteria at certain concentrations enables BV to be diagnosed with positive predictiveness greater than 95% and in particular negative predictiveness greater than 99%.

Also, during BV, bacteria of the Lactobacillus sp. type normally present in the vagina have a diminished concentration, and it has been demonstrated according to the present invention that, past a certain threshold concentration, quantification of these Lactobacillus sp. bacteria allows BV to be reliably diagnosed. Finally, it has been demonstrated that a change in the ratio of the Lactobacillus sp. concentrations to A. vaginae plus G. vaginalis concentrations enables the course of BV to be reliably assessed.

More specifically, the present invention provides a method for in vitro diagnosis and follow-up of the vaginal bacterial flora status in relation to the presence of bacterial vaginosis, and for monitoring its treatment where applicable, characterized in that:

1) the concentrations of the bacteria Atopobium vaginae and Gardnerella vaginalis are quantified by

determining the concentrations of the specific sequences of said bacteria Atopobium vaginae and Gardnerella vaginalis present in a single copy in the DNA of said bacteria Atopobium vaginae and Gardnerella vaginalis, and of a specific sequence of a human gene present in all biological specimens containing human cells, in the DNA extracted from a vaginal discharge specimen from a patient, said specific sequences being less than 150 nucleotides in size,

enzymatic co-amplification of the PCR type of said specific sequences contained, on the one hand, in said DNA extracted from the specimen and, on the other hand, in samples of synthetic DNA fragments including each of said specific sequences of said bacteria and said specific sequence of a human gene present in all biological human cell specimens, said samples serving as calibration standards for quantifying the DNA,

detection and quantification of said amplified fragments being carried out with the aid of probes labeled with sequences different from those of the amplification primers for each of said specific sequences of said bacteria Atopobium vaginae and Gardnerella vaginalis and said specific sequence of a human gene present in all biological human cell specimens, and

2) determining the presence of bacterial vaginosis or failure of the on-going treatment if the concentrations of DNA fragments of said two specific sequences of the bacteria Atopobium vaginae and Gardnerella vaginalis in a specimen of the patient's vaginal discharge containing at least 10⁴ human cells/mL are such that at least one of the following two conditions a) and b) is met:

a) the concentration Ca of said DNA fragment of Atopobium vaginae is greater than or equal to 10⁸ copies/mL, and

b) the concentration Cg of said DNA fragment of Gardnerella vaginalis is greater than or equal to 10⁹ copies/mL.

A concentration of the bacterium Atopobium vaginae greater than or equal to the threshold of 108 allows about 90% of vaginoses to be detected. Quantification of the bacterium Gardnerella vaginalis can be additionally used if the concentration of Atopobium vaginae is less than the threshold of 10⁸ bacteria/mL, to detect vaginosis, because the detection threshold of G. vaginalis greater than or equal to 10⁹ bacteria/mL would by itself enable only about half the cases of vaginosis to be detected. This is why, according to the present invention, it is necessary to quantify the DNA concentrations for both bacteria.

Also, it is noted that development of bacterial vaginosis is confirmed if the Ca, Cg, and Cl concentrations of at least three fragments of specific sequences present in a single copy in the DNA of bacteria A. vaginae (Ca), G. vaginalis (Cg), and Lactobacillus sp. (Cl) in the DNA extracted from a patient vaginal discharge specimen are such that the ratio between the concentrations Cl/(Ca+Cg) decreases between the two specimens sampled sequentially in time at a sufficient time interval, preferably at least one month.

“Development of a vaginosis” is understood here to be worsening of an already-detected vaginosis or, in certain cases, the risk of a vaginosis appearing, i.e., an imbalance or abnormality of the vaginal flora that could become pathological.

Likewise, failure of on-going treatment as a function of the concentrations of the specific sequences present in a single copy in the DNA of the bacteria is confirmed if the ratio between the Cl/(Ca+Cg) concentrations decreases or does not increase between the two specimens sampled sequentially in time at a sufficient time interval, preferably at least one month.

More particularly, a method is created wherein:

a) in step 1), the Lactobacillus sp. including at least the bacteria Lactobacillus acidophilus, Lactobacillus crispatus, Lactobacillus jenseneii, Lactobacillus gasseri and Lactobacillus finers are additionally quantified,

by additionally determining the concentration of a specific sequence of said Lactobacillus sp. bacteria, said specific sequence of Lactobacillus sp. being present in a single copy in the DNA of said Lactobacillus sp. bacteria, and being less than 150 nucleotides in size,

by additional enzymatic co-amplification of the PCR type of said specific sequence of Lactobacillus sp. contained, on the one hand, in said DNA extracted from the specimen and, on the other hand, in a specimen of synthetic DNA fragments including, additionally, said specific sequence of said Lactobacillus sp. bacteria, with said synthetic DNA fragment including said specific sequence of said Lactobacillus sp. bacteria serving as a quantification reference standard,

detection and quantification of said amplified fragments being accomplished with the aid of labeled probes with sequences different from those of the amplification primers for each of said specific sequences of said Atopobium vaginae, Gardnerella vaginalis, and Lactobacillus sp. bacteria and said specific sequence of a human gene present in any biological specimen containing human cells, and

b) in step 2), bacterial vaginosis is determined if, additionally, the Cl concentration of a specific DNA fragment of said Lactobacillus sp. bacteria is less than or equal to 10⁸ copies/mL, preferably less than or equal to 10⁷ copies/mL.

According to the present invention, the bacterial concentration of the Lactobacillus sp. genus is not sufficient to conclude that bacterial vaginosis is present, but is a supplement or confirmation in cases where A. vaginae and G. vaginalis concentrations are combined.

Preferably, bacterial vaginosis is determined if said concentrations are such that the following three conditions are met:

a—Concentration Ca of said DNA fragment of a specific sequence of Atopobium vaginae is greater than or equal to 10⁸ copies/mL,

b—Concentration Cg of said DNA fragment of a specific sequence of Gardnerella vaginalis is greater than or equal to 10⁹ copies/mL, and

c—Concentration Cl of said DNA fragment of a specific sequence of Lactobacillus sp. is less than or equal to 10⁷ copies/mL.

Advantageously, said Ca, Cg, or Cl concentrations are determined by real-time PCR type enzymatic amplification and quantification of the DNA of said DNA fragments of specific sequences of the bacteria Atopobium vaginae, Gardnerella vaginalis and, where applicable, Lactobacillus sp, as well as, preferably, a human DNA fragment present in any human biological specimen containing cells.

Preferably, said specific sequences of said bacteria Atopobium vaginae, Gardnerella vaginalis, and, where applicable, Lactobacillus sp. are 70 to 150 nucleotides in size, preferably 90 to 120 nucleotides.

Still more preferably, real-time PCR amplification and quantification reactions are performed by using hydrolysis probes specific to each of said specific sequences of said bacteria and specific sequence of a human gene present in any biological specimen containing human cells, in the specimen to be tested.

The real-time PCR technique consists of classical PCR using forward and reverse sequence primers, and includes detection of the amplified product based on measuring the fluorescence emission proportional to the quantity of amplified genes with a so-called “hydrolysis” probe. For this purpose, said probe is labeled with a florescence emitter or fluorophore at 5′ and an agent blocking fluorescence emission at 3′. This blocking agent absorbs the fluorescence emitted when the fluorophore and the blocking agent are close together. When the fluorophore and the blocking agent are separated, the fluorescence emission is no longer absorbed by the blocking agent. When it passes, the Taq polymerase causes hydrolysis of the probe and hence release of the nucleotides and fluorophore in solution. The fluorescence emission will thus be proportional to the amplifiate number. The principle of real-time PCR is based on the ability of Taq polymerase during the elongation step to hydrolyze a hybridized probe on the DNA to be copied, this hydrolysis enabling fluorescence emission, which enables quantification. During the same reaction, two different targets can be quantified by introducing into the reaction mixture two primers and one probe directed at a first target, and two other primers and probe directed at the other target. The two probes are labeled with different fluorophores.

Still more preferably, a large synthetic DNA fragment serving as a reference standard for DNA quantification is used, said large synthetic DNA fragment grouping said specific sequences of each of said bacteria whose concentrations are quantified, and said specific human DNA sequence of human cells. The presence of several molecular targets on a given nucleic fragment enables different targets to be quantified in a given specimen and enables them to be co-quantified homogenously; the quantification, by using the same reference range of several molecular species, enables the effectiveness of various PCR reactions to be compared with each other and distinguished from each other over time, avoiding bias linked to the positive control.

“Specific sequence of said bacterium” is understood to be a sequence of the genome of said bacterium that is found in no other living organism genome.

“DNA fragment” is understood to be a DNA fragment or oligonucleotide whose sequences are written below in the 5′→3′ direction.

More particularly, said specific sequences of said bacteria include:

for the Atopobium vaginae bacterium: the fragment of positions 248 to 334 of the 16S ribosomal RNA gene, GenBank reference AY 738658.1,

for the Gardnerella vaginalis bacterium, the fragment of positions 981 to 1072 of the Cpn 60 gene of the chaperone protein 60 kDa, GenBank reference AF 240579.3, and

for the Lactobacillus sp. bacterium, a sequence common to the bacteria Lactobacillus crispatus, Lactobacillus jenseneii, Lactobacillus gasseri, Lactobacillus iners and Lactobacillus acidophilus in the tuf gene coding for the elongation factor at positions 253 to 343 of the gene with GenBank reference AY 562191.1.

Still more particularly, said specific sequences of said bacteria are the following sequences, including probe sequences (underlined) framed by primer sequences (boldfaced) or their reverse and complementary sequences for the anti-sense primers:

for Atopobium vaginae:

Seq. No. 2 = 5′-CCCTATCCGCTCCTGATACCGGCAGGCTTGAGTC
TGGTAGGGGAAGATGGAATTCCAAGTGTGAAATGCGCAGATATTTGG-3′

for Gardnerella vaginalis:

Seq. No. 3 = 5′-CGCATCTGCTAAGGATGTTGAAACATCGTTTAAG
GCTACTGCAACTATTTCTGCAGCAGATCCTGAAGTTGGCGAAAAGATT
GCTG-3′

for Lactobacillus sp.

Seq. No. 4 = 5′-TACATCCCAACTCCAGAACGTGATACTGACAAGC
CATTCTTAATGCAGTTGAAGACGTATTTACTATCACTGGTCGTGGTAC
TGTTGCTT-3′

Advantageously, the human cells present in said specimen, particularly in the DNA obtained from the vaginal specimen to be tested, are quantified as a control for sampling richness, quality of DNA extraction, and potential presence of PCR reaction inhibitors. For this purpose, the number of copies of a human gene present in any human biological specimen containing cells is quantified, particularly the number of copies of the human albumin gene. Quantification of the human albumin gene thus serves as an internal control to attest to the quality and richness of the specimen. Moreover, during patient follow-up, this quantification is a normalization means between two specimens taken at different times. This is because calculating the number of microorganisms per million human cells enables a rigorous inter-specimen comparison to be made. The albumin gene is present only in two copies in the human cells, and measuring the signal of this sequence leads to quantification of the number of initial human cells in the specimen. A number of cells less than or equal to 50 per 5 μl of sample (10⁴ cells/mL), or a quantity of albumin DNA less than or equal to 10²/5 μL, causes the specimen to be rejected due to an insufficient quantity.

Due to the variability in specimen quality, transportation, and preservation, the present invention thus contains a molecular quality control enabling the diagnosis to be systematized with reliable quantification.

Quantification of the cells also enables the absence of PCR reaction limiters to be detected: When the specimen is tested by PCR at various dilutions, the amount of albumin detected increases when the dilutions are increased in the presence of limiters, while it decreases when the dilutions are increased in the absence of PCR reaction limiters.

More particularly, quantification of the human DNA contained in said sample is done, and said large DNA fragment also includes a specific human DNA sequence in the test specimen such as a specific albumin sequence.

Still more particularly, said specific human DNA sequence in the test specimen includes the fragment of positions 16283-16423 of exon 12 of the human albumin gene with GenBank reference M12523.1 with the following sequence or complementary sequence listing:

Seq.No.1=5′-GCTGTCATCTCTTGTGGGCTGTAATCATCGTTTAA GAGTAATATTGCAAAACCTGTCATGCCCACACAAATCTCTCCCTGGCAT TGTTGTCTTTGCAGATGTCAGTGAAAGAGAACCAGCAGCTCCCATGAG TTT-3′, preferably a sequence Seq. No. 1 modified by insertion of a cleavage site, particularly the XhoI site (deleted sequence in parentheses) outside the sequences corresponding to the primers (boldfaced sequences) and the probe sequence (underlined sequence) such as sequence Seq. No. 17.

Again advantageously, amplification and quantification reactions are accomplished by using sets of hydrolysis primers and probes that are specific to each of said test bacteria, and where applicable a specific sequence of human DNA in the test specimen, such as a specific human albumin sequence, and said specific sequence includes a probe sequence surrounded by sequences that can serve as the primer in a PCR type amplification reaction of said specific sequences.

“Probe” is understood here to be an oligonucleotide, preferably 20 to 30 nucleotides, hybridizing specifically with said specific sequence and hence enabling it to be detected and quantified specifically by measuring the increased fluorescence associated with the PCR reaction.

The probe enables the amplified specific DNA to be detected and quantified by comparing the signal intensity with that of the quantification standard.

“Primer” is understood here to be an oligonucleotide preferably with 15 to 25 nucleotides which hybridizes specifically with one of the two ends of the sequence that the polymerase DNA will amplify in the PCR reaction.

More particularly, sets of primers and probes are used, chosen where applicable from the following sequences of the sequence listing attached to the present description, or their complementary sequences:

for Atopobium vaginae:

| | |
|---|---|
| Primer 5′: Seq. No. 5 = | 5′-CCCTATCCGCTCCTGATACC-3′ |
| Primer 3′: Seq. No. 6 = | 5′-CCAAATATCTGCGCATTTCA-3′ |
| Probe: Seq. No. 7 = | 5′-GCAGGCTTGAGTCTGGTAGGGG |
| | A-3′ |

for Gardnerella vaginalis:

| | |
|---|---|
| Primer 5′: Seq. No. 8 = | 5′-CGCATCTGCTAAGGATGTTG-3′ |
| Primer 3′: Seq. No. 9 = | 5′-CAGCAATCTTTTCGCCAACT-3′ |
| Probe: Seq. No. 10 = | 5′-TGCAACTATTTCTGCAGCAGATC |
| | C-3′ |

for Lactobacillus sp.:

| | |
|---|---|
| Primer 5′: Seq. No. 11 = | 5′-TACATCCCAACTCCAGAACG-3′ |
| Primer 3′: Seq. No. 12 = | 5′-AAGCAACAGTACCACGACCA-3′ |
| Probe: Seq. No. 13 = | 5′-TGACAAGCCATTCTTAATGC |
| | A-3′, |

for human albumin:

| | |
|---|---|
| Primer 5′: Seq. No. 14 = | 5′-GCTGTCATCTCTTGTGGGCTG |
| | T-3′ |
| Primer 3′: Seq. No. 15 = | 5′-AAACTCATGGGAGCTGCTGGTT |
| | C-3′ |
| Probe: Seq. No. 16 = | 5′-CCTGTCATGCCCACACAAATCTC |
| | TCC-3′ |

Preferably, said large synthetic DNA fragment constituting the DNA of the standard reference specimen for quantification is inserted into a plasmid.

In these DNA quantification methods, it is important to know whether a positive reaction is due to contamination by the recombinant plasmid used as a quantification standard or as a positive control. To solve this problem, a restriction enzyme cleavage site is advantageously introduced into at least one of the synthetic molecular targets. This site is absent from the natural sequence. Hence, by enzymatic cleavage and analysis of the fragment amplified on agarose gel, or using a real-time PCR probe that specifically recognizes the restriction site, one can detect the presence of any contaminating plasmid.

Thus, more particularly, the following steps can be implemented, wherein:

1) a PCR type enzymatic amplification reaction is conducted of the DNA of at least one said specific sequence of at least one of said agents, in the DNA extracted from said test specimens and in the DNA of the standard reference specimen, with the aid of at least one set of primers able to amplify both at least said authentic specific sequence and said modified specific sequence,

2) verification is made of whether any DNA amplifiates extracted from said test specimens include a said specific sequence, and

3) any false positives coming from any contamination of said specimens to be tested by DNA from the standard reference sample are detected, by at least one of the following steps:

3a) enzymatic digestion of the PCR product obtained with an enzyme corresponding to the cleavage site and analysis on agarose gel of the digestion product by comparison with the PCR product not digested by the restriction enzyme.

If the digested fragment comes from amplification of the molecular target inserted into the control plasma, it contains the restriction site, and will be smaller than the undigested fragment.

3b) a real-time PCR type reaction is performed with forward and reverse primers of one of the molecular targets, and a specific probe of said exogenous sequence containing the restriction site.

Only one fragment coming from the control plasmid and containing the exogenous sequence can be amplified.

More particularly, a said specific sequence of the human DNA in the test specimen is used, which includes the fragment of positions 16283-16423 of exon 12 of the human albumin gene GenBank reference M12523.1 modified by insertion of a cleavage site, particularly site XhoI, outside the sequences corresponding to the primers (boldfaced sequences) and the probe sequence (underlined sequence) of the following sequence listing:

Seq.No.17=5′-GCTGTCATCTCTTGTGGGCTGTAATCATCGT(CT CGAG)TTAAGAGTAATATTGCAAAACCTGTCATGCCCACACAAATCTCT CCCTGGCATTGTTGTCTTTGCAGATGTCAGTGAAAGAGAACCAGCAGC TCCCATGAGTTT-3′, including site XhoI (in parentheses) outside the sequences corresponding to the primers (boldfaced sequences) and the probe sequence (underlined sequence).

More advantageously, a plurality of PCR enzymatic amplification reactions is performed, simultaneously or non-simultaneously with each of said specific sequences of said bacteria with the same large fragment of standard synthetic DNA, with the aid of a plurality of different sets of specific primers of each of the various said specific sequences of each of said bacteria, whereby the sequences of the various primers do not intersect each other between the said various bacteria and said primers can be used in an enzymatic amplification reaction performed according to the same protocol, particularly at the same hybridization temperature.

Various methods are known for constructing a large fragment of chimera DNA combining several fragments, particularly fragments of different origins, especially the method described in FR 2 882 063 wherein a first large double-stranded synthetic DNA fragment is prepared in a given sequence comprising a chain in a particular order of a plurality of n second small contiguous synthetic DNA fragments, consisting essentially of dimerization of a plurality of n oligonucleotides by enzymatic amplification using a heat-resistant polymerase enzyme including:

a first step consisting of a PCR-type amplification reaction of nucleic acids in the presence of a said polymerase enzyme, of a series of n oligonucleotides with given sequences, without exogenous primers, including a series of cycles under temperature conditions enabling said oligonucleotides to be hybridized, followed by elongation of the complex obtained, designed to place said oligonucleotides end to end in a given order, the sequences of said oligonucleotides corresponding sequentially and alternately to the sense and anti-sense sequences of the various said second synthetic fragments, and each said oligonucleotide containing, in its 5′ and 3′ regions, sequences complementary to those of the following and preceding oligonucleotides, where applicable, and

a second amplification step with the aid of specific primers of the 5′ and 3′ ends of said forward strand of said first large synthetic fragment to be prepared, enabling identical copies of said first large fragment to be produced.

Hence, this technique is based on the use and mastery of a PCR artifact, which consists of hybridizing primers among each other (dimerization of primers). This phenomenon is observed in the case where the PCR conditions, particularly temperature, are unsuitable and the primers contain partially complementary sequences.

The construction technique thus consists of choosing, from the target sequences, the oligonucleotide sequences with alternating forward oligonucleotide sequences (“sense”) and reverse (or “anti-sense” oligonucleotide sequences. To make end-to-end placement of these oligonucleotides possible, one is careful to introduce, at 3′ of the sequence of an oligonucleotide, a nucleotide sequence complementing the first nucleotides of the following oligonucleotide. These oligonucleotides will be hybridized by their complementary parts and, because of polymerase activity, for example of Taq polymerase, synthesis of 5′ into 3′ is accomplished to obtain double-stranded fragments. The final (assembled) fragment is synthesized by PCR using a pair of forward and reverse primers corresponding to the sequences of the ends of the first large fragment of synthetic bicatenary DNA desired.

As mentioned above, advantageously, said large fragments of synthetic DNA are advantageously inserted into a plasmid.

This generic construction technique of a synthetic nucleotide fragment allows several molecular targets of interest to be placed in contiguity. This is a simple, rapid, and reliable method, and does not require cumbersome and expensive equipment.

The present invention also relates to a diagnostic kit used for implementing a vaginosis diagnosis and follow-up method according to the invention, characterized by including:

samples of standard reference DNA at a known concentration including said specific sequences of each of said bacteria as defined above, preferably one said specific sequence of human DNA as defined above, and more preferably one said large fragment of synthetic DNA including said specific sequences of each of said bacteria as defined above, and preferably one specific sequence of human DNA as defined above, as well as

said sets of specific primers of said modified synthetic DNA fragments specific to said bacteria, and more preferably, said probes as defined above, and

reagents for implementing a PCR-type DNA amplification reaction.

### BRIEF DESCRIPTION OF THE DRAWINGS

Other features of the present invention will appear from the detailed description below of various embodiments with reference to the sequence listing and to FIGS. 1 to 5.

FIG. 1A represents the analysis of microbial loads of 20 bacterial vaginoses defined by the Nugent score and quantified by real-time PCR according to the present invention.

FIG. 1B represents the analysis of microbial loads of 167 normal flora defined by the Nugent score and quantified by real-time PCR according to the present invention.

FIG. 2 represents the analysis of microbial loads of 44 intermediate flora defined by the Nugent score and quantified by real-time PCR according to the present invention.

FIG. 3 shows 25 bacterial vaginoses identified by molecular criteria and quantified according to the present invention from the group of 44 intermediate flora defined by the Nugent score.

FIG. 4 presents 19 normal flora identified by molecular criteria and quantified according to the present invention after application of the bacterial vaginosis molecular criteria to the group of 44 intermediate flora identified by the Nugent score.

FIG. 5 is a plasmid construction diagram that presents theoretical construction diagrams of an insert by double PCR and the principle of constructing an insert with 6 oligonucleotides.

### DETAILED DESCRIPTION OF EXEMPLARY EMBODIMENTS

### I. I. Patients, Materials, and Methods

I.1 Obtaining and Transporting Vaginal Specimens

The pregnant women whose pregnancies were followed were recruited at the La Conception Hospital in Marseille. An informed consent was the pre-condition for inclusion. The specimens were taken from the posterior vaginal fornix with an unlubricated sterile speculum without antiseptic. Four samples were taken from each woman: two samples by a cotton swab placed in a dry tube (Copan innovation®, Brescia, Italy) and two samples by cytobrush (Scrinet® 5.5 mm, C.C.D. International, Paris, France). One standard cotton swab was used fresh for bacterial culturing. A second swab was placed in a specific transport medium (R1 Urea-Arginine LYO 2, BioMerieux SA, Marcy l'Etoile, France) to look for genital mycoplasmas (M. hominis and M. urealyticum). One cytobrush was used for smearing on a slide and Gram staining. A second cytobrush to extract the DNA for quantification by molecular amplification was carried in 500 μL of MEM transport medium (Minimum Essential Medium, Invitrogen Life Technologies, Carlsbad, Calif., USA). It was frozen at −80° C. from the time it arrived in the laboratory to the time it was used.

I.2 Bacteriological Analysis

I.2.1 Fresh Status

The Trichomonas vaginalis investigation was done by examination while fresh between the slide and cover slip under the optical microscope (10× objective).

I.2 Nugent Score:

I.2.2.1 Gram Staining:

After smearing the specimen onto a glass slide and drying, Gram staining was comprised of: staining with oxalate crystal violet (1 minute), staining with Lugol's solution (1 minute), decoloration with alcohol/acetone and coloration with safranin in solution (BioMerieux). Each step was followed by rinsing in water. Gram staining enables the Nugent score to be established by a semi-quantitative evaluation of three bacterial morphotypes (Table 1). According to the score, three vaginal flora are identified: normal flora (NF) for a score of 0 to 3, intermediate flora (IF) for a score of 4 to 6, and BV for a score of over 7.

I.2.2.2 Culturing

The vaginal specimens were seeded onto three culture media: Columbia ANC agar plus 5% sheep's blood (BioMérieux), Chocolate Poly Vitex PVX agar (BioMerieux), CHOC VCAT agar (BioMérieux) incubated at 37° C. for 48 hours. To detect mycoplasmas, the specimens were seeded onto a specific kit (Urea-Arginine LYO 2, BioMérieux) then incubated at 37° C. and inoculated into anaerobic culture media (BioMerieux) for 48 hours.

I.3. Detection and Quantification By Real-Time PCR

I.3.1 DNA Extraction

DNA extraction was done with the QIAmp®DNA Mini Kit (Qiagen®, Courtaboeuf, France). The protocol was modified as follows: incubation for 12 hours at 56° C. of 200 μL of sample per 200 μL of lysis buffer and 20 μL of proteinase K. The lysate was treated according to the manufacturer's recommendations. The DNA was eluted in 100 μL of elution buffer then stored at −20° C.

I.3.2 Development of Real-Time PCR

I.3.2.1 Choice of Molecular Targets

Analysis of the data in the literature and the sequences deposited at the GenBank site gave information on the sequences available for each of the target microorganisms. The specificity of the probes and fragments of target sequences of each chosen microorganism were tested for specificity at the NBCI website.

The inventors chose the targets from all the potential vaginosis agents, including agents whose role was uncertain. Surprisingly, the most significant target Atopobium vaginae was not considered to be an essential agent.

The selected targets were located on the gene sequence coding for the 60 KDa chaperone protein (Cpn60) for G. vaginalis and M. curtisii, on that of 16S RNA for M. mulieris and A. vaginae, the fts Y sequence for M. hominis, the urease sequence for U. urealyticum, and the topoisomerase III gene for C. albicans. For the Lactobacillus sp., target, a sequence common to: Lactobacillus crispatus, Lactobacillus jensenii, Lactobacillus gasseri, Lactobacillus iners and Lactobacillus acidophilus was chosen; it is located on the gene coding for the elongation factor tu (tuf). A sequence located in exon 12 of the human albumin gene was chosen to attest for the presence and quantity of DNA in the test specimen.

I.3.2.2 Choices of Probes and Primers

For each microorganism studied (Lactobacillus sp., G. vaginalis, M. curtisii, M. mulieris, U. urealyticum, M. hominis, A. vaginae, C. albicans) and human albumin, a probe, and a sense plus anti-sense primer pair were chosen from the target sequences defined above using the Primer 3® program. The primers and probes are listed below. Each primer and probe were analyzed at the NBCI website to ensure their specificity in silico. In the listing below, the “sense primer” and “probe” sequences are written in the 5′→3′ direction, and the “anti-sense primer” sequences are written in the reverse complementary direction 5′→3′.

Molecular target and nucleotide sequence of the primer pair and probe for each microorganism studied.

| Micro- | | |
|---|---|---|
| organism | Target | Nucleotide Sequences |
| M. curtisii | Cpn 60 | Sense primer: SEQ ID NO: 29 |
| | | TGGAAAAGGTGGGTCAAGAG |
| | | Anti-sense primer: SEQ ID NO: 30 |
| | | AAACGCATACCTTCGGTGAC |
| | | Probe: FAM-SEQ ID NO: 31 |
| | | GGCGTCATCACCGTGGAAGAA-TAMRA |
| M. mulieris | 16S ribosomal | Sense primer: SEQ ID NO: 32 |
| | RNA | ATGGATATGCGTGTGGATGG |
| | | Anti-sense primer: SEQ ID NO: 33 |
| | | CCAGGCATGTAAGCCCAAAC |
| | | Probe: VIC-SEQ ID NO: 34 |
| | | TTTTGGGTGGGGGCGCTA-TAMRA |
| G. vaginalis | Cpn 60 | Sense primer: SEQ ID NO: 8 |
| | | CGCATCTGCTAAGGATGTTG |
| | | Anti-sense primer: SEQ ID NO: 9 |
| | | CAGCAATCTTTTCGCCAACT |
| | | Probe: VIC-SEQ ID NO: 10 |
| | | TGCAACTATTTCTGCAGCAGATCC-TAMRA |
| Lactobacillus | Tuf | Sense primer: SEQ ID NO: 11 |
| sp. | | TACATCCCAACTCCAGAACG |
| | | Anti-sense primer: SEQ ID NO: 12 |
| | | AAGCAACAGTACCACGACCA |
| | | Probe: FAM-SEQ ID NO: 13 |
| | | TGACAAGCCATTCTTAATGCA-TAMRA |
| U. urealyticum | Urease | Sense primer: SEQ ID NO: 35 |
| | | ACTGGTGACCGTCCTATCCA |
| | | Anti-sense primer: SEQ ID NO: 36 |
| | | CCTGATGGAATATCGAAACGA |
| | | Probe: VIC-SEQ ID NO: 37 |
| | | TGAAAAGGAAACGAAGACAAAGA- |
| | | TAMRA |
| M. hominis | fts Y | Sense primer: SEQ ID NO: 38 |
| | | ATTGATTGCTGCAGGTGATACA |
| | | Anti-sense primer: SEQ ID NO: 39 |
| | | GGTGTTACAATATCAGCCCCAAC |
| | | Probe: FAM-SEQ ID NO: 40 |
| | | AGAGCAGCGGCAGTTGAA-TAMRA |
| A. vaginae | 16S ribosomal | Sense primer: SEQ ID NO: 5 |
| | RNA | CCCTATCCGCTCCTGATACC |
| | | Anti-sense primer: SEQ ID NO: 6 |
| | | CCAAATATCTGCGCATTTCA |
| | | Probe: VIC-SEQ ID NO: 7 |
| | | GCAGGCTTGAGTCTGGTAGGGGA-TAMRA |
| C. albicans | Topoisomerase 3 | Sense primer: SEQ ID NO: 41 |
| | | CAACGCCAACGAAGACAAG |
| | | Anti-sense primer: SEQ ID NO: 42 |
| | | CCAGCTTTGTTTGCATCAAC |
| | | Probe: FAM-SEQ ID NO: 43 |
| | | AAAGCCGATGGTAGTAGAAAACTGC- |
| | | TAMRA |
| Human | Exon 12 | Sense primer: SEQ ID NO: 14 |
| albumin | | GCTGTCATCTCTTGTGGGCTGT |
| | | Anti-sense primer: SEQ ID NO: 15 |
| | | AAACTCATGGGAGCTGCTGGTTC |
| | | Probe: FAM-SEQ ID NO: 16 |
| | | CCTGTCATGCCCACACAAATCTCTCC- |
| | | TAMRA |

In order to perform real-time dual recognition PCRs (duplex PCRs), recognition pairs of microorganisms were chosen arbitrarily: one of the probes was labeled FAM and the other, VIC. Four PCR pairs were defined in this way.
Lactobacillus sp. (FAM) and G. vaginalis (VIC)
M. curtisii (FAM) and M. mulieris (VIC)
U. urealyticum (VIC) and human albumin (FAM)
A. vaginae (VIC) and C. albicans (FAM)
Mycoplasma hominis (FAM) were to be quantified alone.

The primers were synthesized by Eurogentec® (Seraing, Belgium), and the probes, by Applied Biosystems (Warrington, Cheshire, UK).

I.3.2.3 Specificity Test For the Various Strains Tested

To test the specificity of the primers and probes, the DNA extracted from the reference bacteria strains (L. acidophilus CIP 104464, A. vaginae CIP 106431, G. vaginalis CIP 103660, M. curtisii subsp. holmesii ATCC 35242, M. mulieris ATCC 35239, C. albicans UMIP 1180.79, U. urealyticum CIP 103755 and M. hominis CIP 103715) was used in three dilutions (1:10, 1:100, and 1:1000) for developing real-time PCRs (determination of relative primer and probe quantities, specificities, cross reactivities). Each strain was tested with the primers and the specific probe, but also with the probes and primers of the 7 other microorganisms and those of human albumin.

I.3.2.4 Development of Duplex PCRs

The CT (cycle threshold) values were measured. The CT is the intersection point between the baseline of the reaction and the logarithmic curve representing the amplification. This CT value corresponds to the number of amplification cycles necessary for amplification to begin. It is related to the concentration of the nucleic product to be quantified: the higher the concentration, the lower the CT.

The quantities of primers and probes necessary for obtaining optimal amplification with single and double fluorescence are defined by testing the amplification reaction on a mixture of the bacterial DNA from the 8 reference strains under equimolar conditions, as well as with pure strains, for a final concentration of 1:10 in both cases. The experimental conditions used were those for which the CT values were identical to those obtained with single fluorescence.

I.3.2.5 Specificity Test Versus 40 Untested Strains:

The genomic DNA samples from 40 different bacterial strains (Appendix) excluding the 8 reference bacterial strains were tested in a 1:10 dilution. Care was taken with all these tests to introduce, with each amplification reaction, negative controls containing no DNA or positive controls (mixing of the DNA from the 8 reference strains). Specificity was tested with the four PCR pairs in real time as well as Mycoplasma hominis.

I.4. Construction of Plasmid

The preparation method described in FR 2 882 063 was used.

I.4.1 Quantification Hybrid Fragment Sequence:

The nucleotide sequence of the quantification hybrid fragment was obtained by aligning the PCR target sequences in real time for the 8 microorganisms as well as for those of human albumin. We obtained a hybrid fragment with 931 base pairs, described below.

Overall theoretical sequence of the 939 base pair insert.
Boldfaced sequences=sequences of sense and anti-sense primers
Underlined sequences=specific probe sequences
Black sequences=intercalary sequences
Sequences in parentheses=sequences of Xhol restriction site

Seq. no. 18 = 5′GCCATGGAAAAGGTGGGTCAAGAGGGCGTCATCA
CCGTGGAAGAACATCGTCTCGAGTTAAGGAGCCTCGAAGTCACCGAAG
GTATGCGTTTCATTATGGATATGCGTGTGGATGGATTA(CTCGAG)CT
GCCTGTTTTGGGTGGGGGCGCTATCGGGGTTTGGGCTTACATGCCTGG
CCCTCGCATCTGCTAAGGATGTTGAAACATCGT(CTCGAG)TTAAGGCT
ACTGCAACTATTTCTGCAGCAGATCCTGAAGTTGGCGAAAAGATTGCT
GAATACATCCCAACTCCAGAACGTGATACTGACAAGCCATTCTTAATG
CCAGTTGAAGACGTATTTACTATCACTGGTCGTGGTACTGTTGCTTCT
ATACTGGTGACCGTCCTATCCAAGTTGGATCACATCGT(CTCGAG)TTA
AGAACAAATAGTGCATTAGTATTCTTTGATGAAAAAGGAAACGAAGAC
AAAGAACGTAAAGTTGCTTATGGACGTCGTTTCGATATTCCATCAGGT
AATTGATTGCTGCAGGTGATACATTTAGAGCAGCGGCAGTTGAACAA
TTCATCGT(CTCGAG)TTAAGGAGTTGGGGCTGATATTGTAACACCAA
ACCCCTATCCGCTCCTGATACCGGCAGGCTTGAGTCTGGTAGGGGAA
GATGGAATTCCAAGT(CTCGAG)GTGAAATGCGCAGATATTTGGAAGC
CAACGCCAACGAAGACAAGGCACTTCAAAAAGCCGATGGTAGTAGAA
AACTGCGACATCGT(CTCGAG)TTAAGTTGGTTGATGCAAACAAAGCTG
GTAGTTGCTGTCATCTCTTGTGGGCTGTAATCATCGT(CTCGAG)TTAA
GAGTAATATTGCAAAACCTGTCATGCCCACACAAATCTCTCCCTGGCAT
TGTTGTCTTTGCAGATGTCAGTGAAAGAGAACCAGCAGCTCCCATGAG
TTTGG-3′

I.4.2 Oligonucleotide Sequences in Construction of the Quantification Hybrid Fragment

The hybrid nucleotide fragment sequence was divided into the following 6 consecutive oligonucleotide fragments.

(1) Sense Oligonucleotide 1, 178 Nucleotides: M. curtisii and M. mulieris Sequences

Seq. No. 19 = 5′GCCATGGAAAAGGTGGGTCAAGAGGGCGTCATCA
CCGTGGAAGAACATCGTCTCGAGTTAAGGAGCCTCGAAGTCACCGAAGG
TATGCGTTTCATTATGGATATGCGTGTGGATGGATTACTCGAGCTGCCT
GTTTTGGGTGGGGGCGCTATCGGGGTTTGGGCTTACATGCCTGGCC3′

(2) Anti-Sense Oligonucleotide 2, 183 Nucleotides: G. vaginalis and Lactobacillus sp.

Seq. No. 20 = 5′CGACCAGTGATAGTAAATACGTCTTCAACTGGCA
TTAAGAATGGCTTGTCAGTATCACGTTCTGGAGTTGGGATGTATTCAGCA
ATCTTTTCGCCAACTTCAGGATCTGCTGCAGAAATAGTTGCAGTAGCCTT
AACTCGAGACGATGTTTCAACATCCTTAGCAGATGCGAGGGCCAGGCA
T3′

(3) Sense Oligonucleotide 3, 145 Nucleotides: U. urealyticum

Seq. No. 21 = 5′TCACTGGTCGTGGTACTGTTGCTTCTATACTGGT
GACCGTCCTATCCAAGTTGGATCACATCGTCTCGAGTTAAGAACAAATAG
TGCATTAGTATTCTTTGATGAAAAAGGAAACGAAGACAAAGAACGTAAA
GTTGCTTATGGA3′

(4) Anti-Sense Oligonucleotide 4, 188 Nucleotides: M. hominis

Seq. No. 22 = 5′CTTGGAATTCCATCTTCCCCTACCAGACTCAAGC
CTGCCGGTATCAGGAGCGGATAGGGGTTTGGTGTTACAATATCAGCCCCA
ACTCCTTAACTCGAGACGATGAATTGTTCAACTGCCGCTGCTCTAAATG
TATCACCTGCAGCAATCAATTACCTGATGGAATATCGAAACGACGTCCA
TAAGCA3′

(5) Sense Oligonucleotide 5, 152 Nucleotides: A. vaginae and C. albicans

Seq. No. 23 = 5′GAATTCCAAGTCTCGAGGTGAAATGCGCAGATAT
TTGGAAGCCAACGCCAACGAAGACAAGGCACTTCAAAAAGCCGATGGTAG
TAGAAAACTGCGACATCGTCTCGAGTTAAGTTGGTTGATGCAAACAAA
GCTGGTAGTTGCTGTCAT3′

(6) Anti-Sense Oligonucleotide 6, 147 Nucleotides: Human Albumin

Seq. No. 24 = 5′CCAAACTCATGGGAGCTGCTGGTTCTCTTTCACT
GACATCTGCAAAGACAACAATGCCAGGGAGAGATTTGTGTGGGCATGACA
GGTTTTGCAATATTACTCTTAACTCGAGACGATGATTACAGCCCACAAG
AGATGACAGCAA3′

To ensure continuity of adjacent oligonucleotides, 10 additional nucleotides at the 3′ end on the upstream oligonucleotide were added to the 5′ end of the downstream oligonucleotide. The sizes of the 6 oligonucleotide sequences ranged from 155 to 172 nucleotides. The consecutive oligonucleotides were alternatively in forward and reverse sequence. Thus, oligonucleotides 1, 3, and 5 were used in the forward sequencing form while oligonucleotides 2, 4, and 6 were used in the reverse sequencing form.

Sense and anti-sense “construction” primers (below), whose sequences corresponded to those of the 20 nucleotides at 5′ and 3′ of the quantification hybrid fragment, were to be synthesized.

### Construction Primers for Recombinant Plasmid With the Fragment of 939 Base Pairs

Sense Seq. No. 25 = 5′GCCATGGAAAAGGTGGGTC3′
Anti-sense Seq. No. 28 = 5′CCAAACTCATGGGAGCTGCT3′

The oligonucleotides and primers were synthesized by Eurogentec®.

I.4.3 Construction of Hybrid Fragment:

The double-stranded nucleotide fragment was constructed by an amplification reaction thanks to the complementarity of the ends of two adjacent oligonucleotides. Two successive PCRs were necessary.

I.4.3.1 First PCR:

This allowed hybridization of the oligonucleotides by their ends and partial elongation when this is compatible with activity of the Taq polymerase. The 6 oligonucleotides were introduced under equimolar conditions (0.2 mMol), with polymerase buffer 1× MgCl₂ (1.5 mMol), dNTP (0.2 mMol), 0.2 μL of Taq Roche (Roche®), 5 IU/μL, in a reaction volume of The amplification program was: 95° C. 2 min, followed by 40 cycles comprising 94° C. 30 sec (denaturation), 37° C. 1 min (hybridization), 72° C. 1 min 30 sec (elongation).

I.4.3.2 Second PCR:

This allows a PCR fragment containing the expected oligonucleotide groups end-to-end to be obtained (FIG. 5). One μL of amplification product from the first PCR was added to the following reaction mixture: polymerase buffer Hotstar (Qiagen®) 1× MgCl₂ (1.5 mMol), dNTP (0.2 mMol), 0.2 μL of Hotstar (Qiagen®) with 5 IU/μL, and 0.2 mMol of sense and anti-sense construction primers. The PCR program was: 95° C. 15 min, followed by 95° C. 30 sec, 58° C. 45 sec, 72° C. 2 min 40 cycles, 72° C. 5 min. The PCR fragment obtained was analyzed on 1.5% B ET agarose gel in TBE buffer 0.5×. If the size of the fragment was the expected size, it was purified using the QIAquick® PCR Purification Kit 250 PCR Qiakit (Qiagen®).

I.4.3.3 Cloning the Insert

Two μL of the fragment obtained previously were introduced into a ligation reaction mixture containing 5 μL of ligase buffer, 1 μL of ligase, and 1 μL of linearized, dephosphorylated plasmid PGEM (kit pGEM®-T Easy Vector System 2 Promega ®, Madison, Wis., USA). The final volume was 10 μL. The ligation product was incubated at 15° C. overnight. Seven μL of the ligation product were mixed with 50 μL of competent cells (Escherichia coli JM 109) kept in ice for 20 minutes then incubated for 1 minute at 42° C. After addition of 950 μL of LB broth (USB®, Cleveland, Ohio, USA), and incubation for 1 h 30 min at 37° C., 500 and 250 μL of this culture medium were smeared on 2 Petri dishes containing LB agar (USB®) with 100 μg/mL of ampicillin. The dishes were incubated overnight at 37° C. The recombinant colonies were deposited both in 50 μL of sterile distilled water and on an LB agar ampicillin Petri dish.

The recombinant E. coli colonies were sampled for PCR analysis: 5 μl of bacterial suspension in distilled water, the pair of M13 primers (10 pm/μL), and the previously described PCR reaction medium. The PCR program was identical to that used for the second construction step. The PCR products obtained were analyzed in 1.5% gel agarose in TBE buffer 0.5×. The fragments of the expected size were purified using the Qiaquick® PCR Purification Kit 250 Qiakit (Qiagen®). They were then sequenced using Big Dye® Terminator V1.1 Cycle Sequencing Kit (Applied Biosystems®, Warrington, UK), 3.2 pmol/μL of sense and anti-sense primers chromatographed on the ABI PRIM 3100 sequencer (Applied Biosystems®). The sequence obtained was compared to the sequence of the expected fragment using the auto-assembler and Sequence Navigator (Applied Biosystems®) programs. One of the recombinant clones, conforming to the expected sequence, was to be produced in large amounts in 100 mL of LB broth ampicillin and purified according to the High Speed Plasmid Midi Kit (Qiagen®) protocol. The purified plasmid was then stored at −20° C. The selected recombinant strain was frozen at −80° C.

I.4.3.4 Obtaining the Plasmid Range

Its concentration was determined by measuring the optical density at 260 nm. For example, let 0.38 be for the initial solution. 1 unit OD corresponds to 50 μL/mL: 0.38×50=19 μg/mL=19×10⁻⁶ g/mL of plasmid in the initial solution. Plasmid size=size of pGEM®-T Easy+fragment size=3015+931=4334 bp, or 8668 bases. The molar mass of one nucleotide is 330 Da (g/mol). The molar mass of the plasmid will be 8668×330=2.860×10⁶ g/mol. The plasmid concentration will be in mol/mL, or 19×10⁻⁶/2.860×10⁶=6.64×10⁻¹² mol/mL. Multiplied by Avogadro's number, we obtained the number of plasmid copies per 1 MmL of solution, namely: 6.64×10⁻¹²×6.023. 10²³=40×10¹¹ copies/mL or 2×10¹⁰ copies/5 μL. A first dilution of the initial 1:2500 plasmid solution enabled the concentration to be adjusted to the first step of the plasmid range, or 10⁷ copies/5 μL of plasmid solution. A dilution cascade in steps of 10 enabled successive steps of the range to be created (of 10⁷ copies to 1 copy per 5 μL of solution).

I.5. Analysis of Specimens

I.5.1 Real-Time Quantitative PCR

The quantification reactions were done by real-time PCR on DNA extracts from vaginal specimens. The following were placed on each reaction plate: 4 negative controls (NTC), the calibration plasmid range (10⁷ to 1 copy par well), and 24 test specimens, pure and diluted to 1:10 and 1:100, to look for inhibitors. The negative controls and the points on the plasmid range were tested in duplicate. For amplification and quantification of the 8 microorganisms and human albumin, 4 PCR plates were run with double fluorescence and one with single fluorescence. For preparation of the reaction mixture, the kit Quantitect Probe PCR Kit (Qiagen®) containing the 2× reaction mixture combining the Taq polymerase and Taq polymerase buffer (Hotstar), dNTP, and dUTP was used. To this reaction mixture were added the sense and anti-sense probes necessary for single or double fluorescence PCR according to the experimental conditions reported and described in Appendix 6, with the test specimens diluted or undiluted; the points on the plasmid range were 5 μL and 0.25 μL of UDG (Uracil DNA glycosylase, 100 Units, Sigma-Aldrich, Lyon, France) was added for a final reaction volume of 25 μL. The PCR reactions were run on the Stratagene® MX 3000P (La Jolla, Calif.). The amplification program was the following: 2 minutes at 50° C., 15 minutes at 95° C., followed by 45 PCR cycles consisting of denaturation at 95° C. for 30 seconds then the hybridization and elongation phase at 60° C. for 1 minute.

I.5.2 Calculation of Bacterial Load

For each vaginal specimen and each microorganism, in order to ensure comparability of the specimens, the bacterial load was defined as follows. The quantification in number of DNA copies from each microorganism per 5 μL of DNA extract was reported as the number of bacteria per 1 mL of initial specimen. The DNA elution volume (100 μL), the volume of the transport medium per initial specimen (200 μL), and the fact that the quantified gene is a single gene must be taken into account. The value obtained for each microorganism in numbers of copies per 5 μL of DNA extract was multiplied by 10² to obtain a concentration in number of bacteria per ml of specimen, then an elution volume of 100 μL of DNA extracted from 200 μL of sample was created.

I.5.3 Statistical Analysis

The statistical analysis of the real-time PCR bacterial quantification data employed the Wilcoxon test and the Mann-Whitney test for one degree of significance p<0.05. For the statistical analysis, all the quantification values were taken into account, including those below the positivity threshold. For quantification values equal to zero, the lowest concentration of the total specimens analyzed was attributed to them. The Wilcoxon statistical test was applied to describe the distribution of each microorganism within each group of flora. The Mann-Whitney test was applied to compare the quantification of each of the 8 microorganisms in the BV and NF group. In order to investigate the molecular criteria for BV, each bacterial quantification threshold (de 10³/mL to 10⁹/mL) was studied for sensitivity, specificity, and positive and negative predictive value, calculated according to the identification by the NF and BV thresholds previously defined by the Nugent score. The quantification thresholds, taken in isolation or combined, having the best sensitivity and specificity were used as molecular criteria for identification of BV.

II. Results

II.1. Description of Population

From June 2005 to April 2006, 204 pregnant women aged 18 to 49 (average age 28.9±6.2) were included. The ethnic origins were North Africa (43%), Europe (42%), South Africa (14%), and other origins (1%). One vaginal specimen was taken from each woman. Seventy-two percent of the specimens were taken in the third trimester, 20% in the second, and 8% in the first trimester of pregnancy. Twenty-one women received bacteriological follow-up, with 2 to 4 specimens being taken. Hence, a total of 231 specimens was analyzed.

II.2. Bacteriology Results

II.2.1 Gram Staining and Establishing Nugent's Score

From the 231 vaginal specimens from 204 women, the Nugent's score identified: 167 NF, 44 IF, and 20 BV. The frequency of vaginal flora abnormalities in the first vaginal specimen for each of the 204 women was 71% for NF (145 cases), 19% for IF (39 cases), and 10% for BV (20 cases). Half of the women with BV were symptomatic. The most frequently observed symptom was abundant vaginal discharge.

II.2.2 Culturing and Fresh Status

Culturing enabled G. vaginalis, C. albicans, M. hominis, U. urealyticum, and Streptococcus agalatiae to be isolated (Table 1). None of the vaginal specimens examined exhibited T. vaginalis on direct examination.

II.3. Molecular Biology Results

II.3.1 Development of Real-Time PCR

When the single and double fluorescence PCRs were developed, no cross reactions were done, with no competition. Similar CT value results were obtained with single and double fluorescence using the pure bacterial strains and the plasmid range.

The optimum quantities of primers and probes necessary for obtaining optimum amplification are presented below.

Quantity of probe and sense and anti-sense primers necessary for the amplification reaction of 5 μL of DNA extract for each microorganism and human albumin.

| | Probe | Sense primer | Anti-sense primer |
|---|---|---|---|
| | (quantity | (quantity | (quantity |
| | expressed in | expressed in | expressed |
| Micro-organism | μL per well) | μL per well) | in μL per well) |
| M. curtisii | 0.25 | 0.5 | 0.5 |
| M. mulieris | 0.25 | 0.5 | 0.5 |
| G. vaginalis | 0.25 | 0.5 | 0.5 |
| Lactobacillus | 0.25 | 0.5 | 0.5 |
| sp. | | | |
| U. urealyticum | 0.25 | 0.5 | 0.5 |
| M. hominis | 0.125 | 0.125 | 0.125 |
| A. vaginae | 0.25 | 0.5 | 0.5 |
| C. albicans | 0.25 | 0.5 | 0.5 |
| Human albumin | 0.125 | 0.125 | 0.125 |

II.3.2 Evaluation of Quantification Technique By Molecular Biology

Bacterial quantification for each specimen was provided by the plasma dilution range. For each amplification reaction, the 8 points on the plasmid range, from 10⁷ copies to 1 copy per 5 μL, were tested. The 10⁷ copies range point was identified early for a CT of about 17. For the 1 copy range point, detection was later with a CT at 37. For all the points on the range, the amplification reaction was linear. The graphic representation is a straight line with a slope of −3.2 to −3.5. This linearity was confirmed by testing the pure strains diluted to 1:10, 1:100, and 1:1000. A positivity threshold was defined above 10 copies (or 10³ bacteria/mL), whatever the type of microorganism considered (Table 2). Statistically, amplification of the “1 copy” point occurs in 75% while it is 100% for the “10 copies” point. Within each amplification plate, each specimen was tested in undiluted solution, and one-tenth and one-hundredth dilutions. Reproducibly, detection of the amplification product followed the dilution step, since a one-tenth dilution corresponds to an increase of 3 CTs. Only the pure solution was considered for calculating the bacterial load. For each specimen, we tested human albumin by real-time PCR. The results obtained were homogenous for the set of 231 samples. The CT values were distributed between 19 and 22 (or 10⁵ to 10⁶ copies of albumin DNA per 5 μL of DNA extracted). No specimen was excluded from the analysis.

II.3.3 Analysis of Data

II.3.3.1 Molecular Description of Bacterial Vaginoses

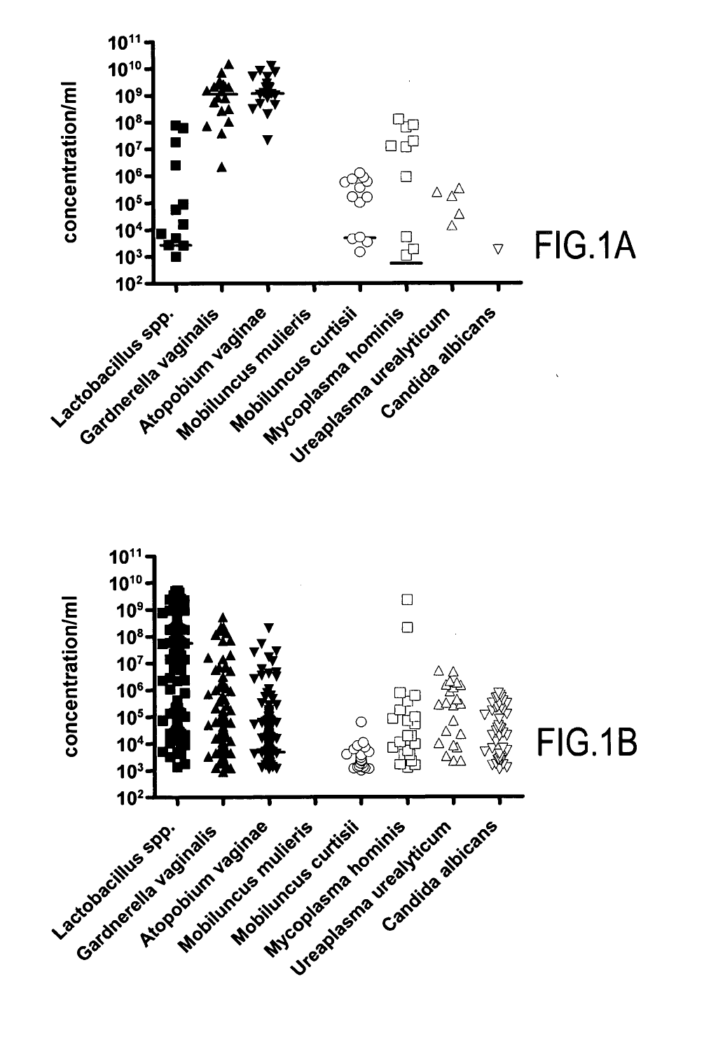

The median concentrations of A. vaginae and G. vaginalis were 1.1×10⁹/mL and 1.2×10⁹/mL respectively, and there was no statistically significant difference with the Wilcoxon test (p=0.3755). These two bacteria were in high concentrations (FIGS. 1A and 3) relative to the other microorganisms whose median concentrations were statistically lower (p=0.0001).

II.3.3.2 Molecular Comparison of Bacterial Vaginoses and Normal Flora

The median concentration of Lactobacillus sp. was significantly lower (p<0.0001) for bacterial vaginoses (median concentration 3×10³/mL) than for the NF (median concentration 5.7×10⁷/mL). On the other hand, the median concentrations of A. vaginae, G. vaginalis, M. curtisii and M. hominis were significantly higher (p<0.0037) for bacterial vaginoses (median concentrations respectively 1×10⁹ /mL; 1.2×10⁹ /mL; 5×10³ /mL and 5.5×10² /mL) than for the NF. For the median concentrations of U. urealyticum and C. albicans, there was no statistically significant difference between the BV and the NF. Finally, M. mulieris was not identified (positivity threshold≧10³/mL) in any of the BVs or any of the NF.

II.3.3.3 Definition of Molecular Criteria for Bacterial Vaginosis

The analysis by quantification threshold for A. vaginae and G. vaginalis taken in isolation has the best criteria for sensitivity, specificity, and positive and negative predictive values for molecular identification of BV and NF defined by the Nugent score (Table 4). The combination of quantification of A. vaginae≧10⁸/mL and/or quantification of G. vaginalis≧10⁹/mL has a sensitivity of 95%, specificity of 99%, positive predictive value (PPV) of 95%, and negative predictive value (NPV) of 99%.

II.3.3.4 Molecular Characterization of Intermediate Flora

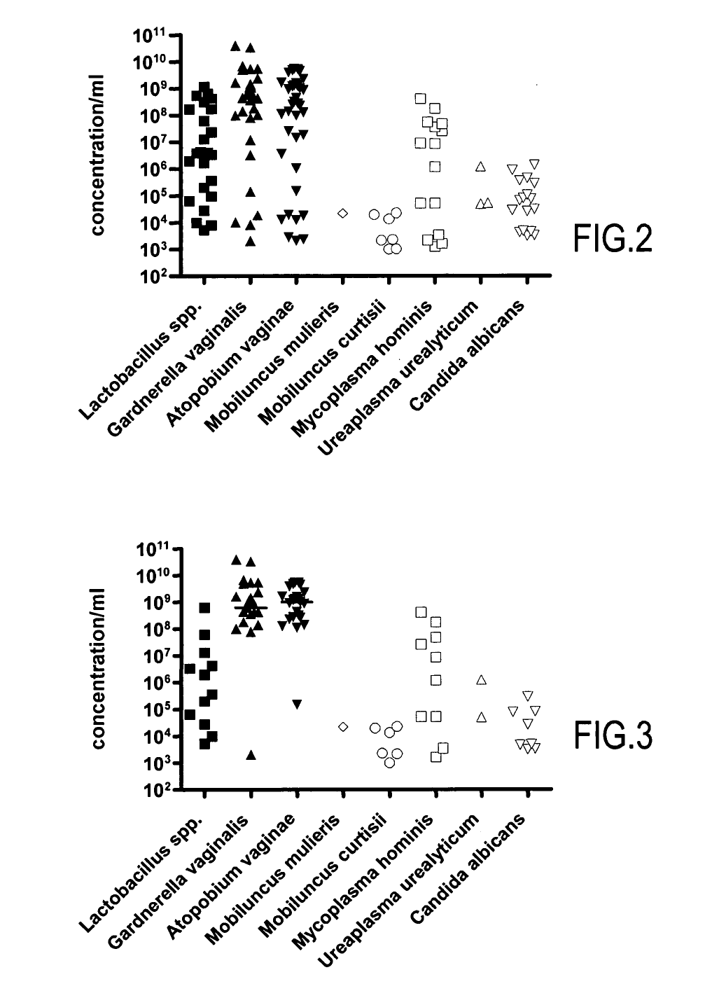

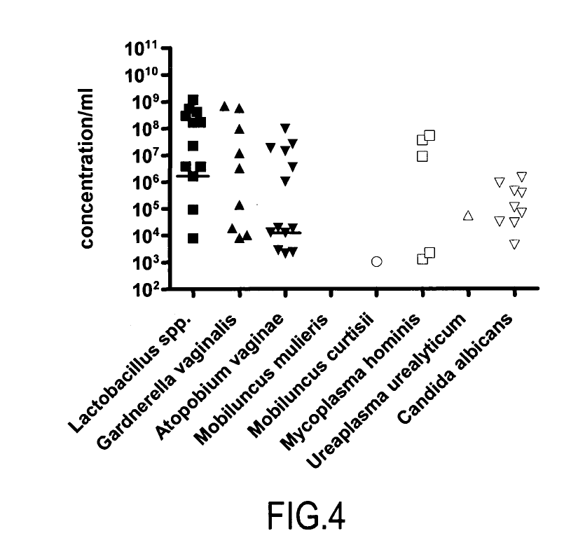

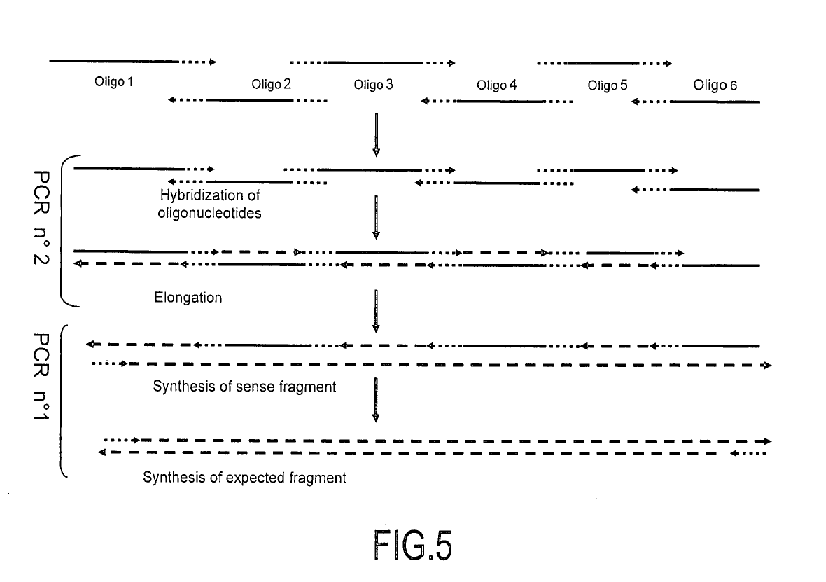

Application to IF of the Nugent score (FIG. 2) of the molecular criteria for BV identification previously defined by quantification of A. vaginae≧10⁸/mL and/or quantification of G. vaginalis≧10⁹/mL, allowed 25 flora to be characterized (57%) that had a BV profile (FIGS. 3) and 19 flora (43%) with an NF profile (FIG. 4).

II 11.3.3.5 Bacteriological follow-up

Eight women with BV or IF by the Nugent score were followed (Table 6). Molecular quantification showed a relapse of a BV not identified by the Nugent score for subject 1. It confirmed disappearance of BV for subjects 7 and 8. It characterized as BV the IF in the Nugent score persisting over a month later for subjects 2 and 5. Finally, it confirmed the normal nature of the vaginal flora followed in subject 3.

III Discussion

The distribution of vaginal flora into NF (71%), BV (10%) and IF (19%) according to the Nugent score conforms to those reported in the literature in France [Goffinet F, EJOGR 2003], Europe [Guise J M, AMJPM 2001], and the United States [Delaney M L, OG 2001]. The original feature of the present invention is establishing a rational tool for vaginal flora identification by combining the specific real-time PCR detection technique with relative quantification by a calibration plasmid.

The most surprising result was for A. vaginae. This bacterium was identified for the first time in 1999 by 16S RNA amplification and sequencing from a vaginal specimen from a healthy subject [Rodriguez J M, IJSB 1999]. In 2003, A. vaginae was isolated by culturing a specimen from a tuboovarian abscess, suggesting that the bacterium played a pathogenic role [Geissdorfer W, JCM 2003]. In 2004, an approach combining 16S rRNA with cloning techniques revealed the bacterium in specimens obtained perioperatively from patients with salpingitis [Hebb J K, JID 2004]. According to the present invention, this bacterium is frequently identified in 19 out of 20 BV (95%) and 115 out of 167 NF (69%). The most discriminating criterion for BV and NF diagnosis is an A. vaginae concentration≧10⁸/mL with a sensitivity of 90% (18 cases out of 20 BV) and specificity of 99% (1 case out of 167 NF).

Since 2004, four studies based on various molecular techniques have shown a possible association between A. vaginae and BV, but none of them used rigorous quantification criteria. First of all, a PCR approach targeting 16S rRNA followed by gel migration showed A. vaginae in only 12 BV out of 20 (60%) and 2 NF out of 24 (8.3%) [Ferris M J, BMCID 2004]. By PCR specifically targeting the 16S rRNA gene of A. vaginae, the DNA of the bacterium was found in 7 out of 9 BV cases (77.8%) and 22 NF out of 112 (19.6%) [Verhelst R, BMCM 2004]. Applying the same technique, A. vaginae DNA was shown in 19 out of 22 BV (86.4%) and 59 NF out of 403 (14.7%) [Verhelst R, BMCM 2005]. Finally, by amplification, cloning, and sequencing techniques, A. vaginae DNA was observed in 26 BV out of 27 (96%) and 9 NF out of 46 (19.5%) [Fredricks D N, NEJ M 2005]. None of these studies quantified the A. vaginae DNA, which is essential in bacterial vaginosis where the bacterial concentration is an important factor in the diagnosis.

A. vaginae is missing from the Nugent score despite its leading role in vaginal flora abnormalities. Its identification by morphological criteria is ill-suited. Its variable morphology in the form of a small Gram-positive coccobacillus (0.6-0.9 μm) sometimes grouped in pairs or short chains camouflages it when it contacts other bacteria, rendering it undetectable [Verhelst R, BMCM 2004]. Its resemblance to Lactobacillus sp. and to streptococci is hence a source of identification error [Rodriguez J M, IJSB 1999].

The description of an association between A. vaginae and G. vaginalis in BV is recent, sparsely documented, and of limited diagnostic scope in the absence of quantification. This association is 87.8% (8 out of 9 BV) [Verhelst R, BMCM 2004] and 90% (20 out of 22 BV) [Zariffard M R, FEMS 2002] by specific PCR that targets the 16S rRNA gene of A. vaginae and G. vaginalis. A recent publication notes the presence of A. vaginae in bacterial vaginoses by a semi-quantitative method [Bradshaw C S, JID 2006].

According to the present invention, this combination of A. vaginae and G. vaginalis is 90% (18 out of 20 BV) but consideration of the quantification of A. vaginae≧10⁸/mL and/or of G. vaginalis≧10⁹/mL with a sensitivity of 95% (19 out of 20 BV) offers the best criteria for specificity (99%), PPV (95%) and NPV (99%) ever to have been achieved for identification of BV. Hence, an A. vaginae concentration of ≧10⁸/mL and/or a G. vaginalis concentration of ≧10⁹/mL is used for molecular diagnosis of BV.

The results according to the present invention suggest that the quantification of G. vaginalis is less discriminating than that of A. vaginae. For Lactobacillus sp., their diminution in BV shown objectively by the Nugent score is confirmed by these results: if we consider a Lactobacillus sp. concentration less than or equal to 10⁷/mL, the sensitivity for diagnosing BV is 100% and the specificity, 44%. Moreover, if we consider a Lactobacillus sp. concentration greater than or equal to 10⁸/mL, we observe 100% specificity in demonstrating normal vaginal flora.

For Mobiluncus spp., despite the position afforded them by the Nugent score, no PCR is positive for M. mulieris. Only M. curtisii is associated with BV. However, its utility as a possible molecular diagnostic criterion remains minor.

Genital mycoplasmas are not taken into account by the Nugent score. They are however identifiable by culturing or molecular biology. A study culturing 445 BV and 2729 NF identified M. hominis as being significantly associated with BV with a prevalence of 29% (129 BV) and U. urealyticum as not being associated with BV despite a prevalence of 56% (253 BV) [Thorsen P, AJOG 1998]. A study on 203 BV and 203 NF targeting M. hominis by real-time PCR suggested involvement of M. hominis in BV [Zariffard M R, FEMS 2002]. However, an identical study on a smaller cohort (5 BV and 16 NF) did not demonstrate this link [Sha B E, JCM 2005]. According to the present invention, only M. hominis correlates with BV, but this correlation is insufficient for proposing this microorganism as a diagnostic criterion for BV.

The results according to the present invention do not show a link between BV and C. albicans as shown by the data in the literature [Thorsen P, AJOG 1998]. This is interesting because while genital carriage of Candida albicans does not increase the risk of prematurity [Cotch M F, AJOG 1998], a recent study [Kiss H, BMJ 2004] showed a reduction in prematurity rate by treatment of C. albicans genital portage.

The originality of the present invention is that for the first time for the first time it provides a diagnostic tool for BV based on quantification of A. vaginae and G. vaginalis. The criteria in current use combine a concentration of A. vaginae≧10⁸/mL and/or a G. vaginalis concentration of ≧10⁹/mL. This diagnostic test has a sensitivity of 95%, a specificity of 99%, a PPV of 95%, and a NPV of 99%. Several methods of reading the PCR results, particularly those in which the ratio and products of the bacterial concentrations are calculated, have been tested so that the most pertinent method could be identified.

One of the most disturbing applications of the molecular biology tool according to the present invention is the one performed on the IF in the Nugent score. We know that the IF, identified solely by the Nugent score, constitutes a non-negligible fraction (8% to 22%) of all the flora identified by the latter [Guerra B, EJOGRB 2006; Goffinet F, EJOGRB 2003; Delaney M L, OG 2001; Libman M D, DMID 2006; Larsson P G, STI 2004] and that it is associated with many uncertainties. Indeed, its interpretation requires caution because we do not know the microbiological reality to which this intermediate flora corresponds. Some authors believe it to be a transitional flora between NF and BV [Ison C A, STI 2002; Guerra B, EJOGRB 2006; Goffinet F, EJOGRB 2003; Ugwumadu A, Lancet 2003; Carey J C, NEJM 2000]. Others consider it to be a heterogenous group including NF and BV [Ison C A, STI 2002; Libman M D, DMID 2006; Larsson P G, STI 2004]. Attempts to characterize the IF in NF or BV are reported in the literature. By applying Amsel's criteria to 13 IF, 12 IF were reclassified as NF and 1 as BV [Icon C A, STI 2002 ]. Using Kopeloff's stain to establish the Nugent score, 69 of the 232 IF (30%) were reclassified as NF or BV [Libman M D, DMID 2006 ]. In this stain, fuchsin replaces the safranin in Gram's stain for better identification of Gram-negative bacteria. Finally, the standardization of the Nugent score criteria as a function of the surface of the optical field of the microscope used enabled 458 out of 1176 IF (39%) to be reclassified as NF or BV [Larsson P G, STI 2004].

The application to IF of the molecular criteria according to the present invention associating an A. vaginae concentration of ≧10⁸/mL and/or a G. vaginalis concentration of ≧10⁹/mL with BV enables this IF to be rationally characterized. Thus, 24 IF (57%) have a profile similar to that of NF. These results thus suggest that the IF are heterogenous in nature, and that the Nugent score is so insensitive that it cannot diagnose over half the instances of BV. Thus, these data confirm the limitations of the Nugent score.

The etiology of BV remains quite mysterious, but for the first time we have an objective quantification tool providing a rational approach to the diagnosis of BV. The singular nature of the present invention is showing both the crucial role of A. vaginae in BV and its utilization by quantification as a main diagnostic criterion for BV. It offers better understanding of the therapeutic problem posed by the relative resistance of A. vaginae to metronidazole, which could in part account for the frequency of relapses after treatment [ANAES 2001; Ferris M J, BMC ID 2004; Secor A M, CNP 1997; Geissdorfer W, JCM 2003; De Backer E, BMC ID 2006]. The molecular criteria used to diagnose BV combine an A. vaginae concentration≧10⁸/mL and/or a G. vaginalis concentration≧10⁹/mL. This molecular tool enables BV to be diagnosed and some IF to be characterized as BV. We can also envisage our molecular tool as being a BV diagnostic tool and a follow-up method for evaluating therapeutic management of BV during pregnancy.

### REFERENCES

ANAES. 2001. Prévention anténatale du risque infectieux bactérien anténatal. Recommandations pour les professionnels de santé. [Prenatal prevention of prenatal bacterial infection risk. Recommendations for healthcare professionals.]

Amsel R, Totten P A, Spiegel C A, Chen K C, Eschenbach D, Holmes K K. Nonspecific vaginitis. Diagnostic criteria and microbial and epidemiologic associations. Am J Med. 1983;74:14-22.

De Backer E, Verhelst R, Verstraelen Hans, Antibiotic susceptibility of Atopobium vaginae. BMC infectious diseases, March 2006.

Bradshaw C S, Morton A N, Hocking J. High recurrence rates of bacterial vaginosis over the course of 12 months after oral metronidazole therapy and factors associated with recurrence. J Infect Dis 2006; 193:1478-86.

Bradshaw C S, Tabrizi S N, Fairley C K, Morton A N, Rudland E, Garland S M. The association of Atopobium vaginae and Gardnerella vaginalis with bacterial vaginosis and recurrence after oral metronidazole therapy. J Infect Dis. 2006; 194:828-36.

Carey J C, Klebanoff M A, Hauth J C, Hillier S L, Thom E A, Ernest J M, Heine R P, Nugent R P, Fischer M L, Leveno K J, Wapner R, Varner M. Metronidazole to prevent preterm delivery in pregnant women with asymptomatic bacterial vaginosis. National Institute of Child Health and Human Development Network of Maternal-Fetal Medicine Units. N Engl J Med. 2000 ;342:534-40.

Cotch M F, Hillier S L, Gibbs R S, Eschenbach D A. Epidemiology and outcomes associated with moderate to heavy Candida colonization during pregnancy. Vaginal Infections and Prematurity Study Group. Am J Obstet Gynecol. 1998;178:374-80.

Delaney M L, Onderdonk A B. Nugent score related to vaginal culture in pregnant women. Obstet Gynecol. 2001;98:79-84.

Ferris M J, Masztal A, Aldridge K E, Fortenberry J D, Fidel P L Jr, Martin D H. Association of Atopobium vaginae, a recently described metronidazole resistant anaerobe, with bacterial vaginosis. BMC Infect Dis. 2004 Feb. 13; 4:5.

Ferris, M. J., A. Masztal, and D. H. Martin. Use of species-directed 16S rRNA gene PCR primers for detection of Atopobium vaginae in patients with bacterial vaginosis. J Clin Microbiol. 2004; 42:5892-4.

Fredricks D N, Fiedler T L, Marrazzo J M. Molecular identification of bacteria associated with bacterial vaginosis. N Engl J Med. 2005; 353:1899-911.

Geissdorfer W, Bohmer C, Pelz K, Schoerner C, Frobenius W, Bogdan C Tuboovarian abscess caused by Atopobium vaginae following transvaginal oocyte recovery. J Clin Microbiol. 2003;41:2788-90.

Goffinet F, Maillard F, Mihoubi N, Kayem G, Papiernik E, Cabrol D, Paul G. Bacterial vaginosis: prevalence and predictive value for premature delivery and neonatal infection in women with preterm labour and intact membranes. Eur J Obstet Gynecol Reprod Biol. 2003;108:146-51.

Guerra B, Ghi T, Quarta S, Morselli-Labate AM, Lazzarotto T, Pilu G, Rizzo N. Pregnancy outcome after early detection of bacterial vaginosis. Eur J Obstet Gynecol Reprod Biol. 2006 Feb. 2.

Guise J M, Mahon S M, Aickin M, Helfand M, Peipert J F, Westhoff C. Screening for bacterial vaginosis in pregnancy. Am J Prey Med. 2001 ;20(3 Suppl):62-72.

Hauth J C, Goldenberg R L, Andrews W W, DuBard M B, Copper R L. Reduced incidence of preterm delivery with metronidazole and erythromycin in women with bacterial vaginosis. N Engl J Med. 1995;333:1732-6.

Hebb J K, Cohen C R, Astete S G, Bukusi E A, Totten P A. Detection of novel organisms associated with salpingitis, by use of 16S rDNA polymerase chain reaction. J Infect Dis. 2004;190:2109-20.

Ison C A, Hay P E. Validation of a simplified grading of Gram stained vaginal smears for use in genitourinary medicine clinics. Sex Transm Infect. 2002;78:413-5.

Kiss H, Petricevic L, Husslein P. Prospective randomised controlled trial of an infection screening programme to reduce the rate of preterm delivery. BMJ. 2004;329:371.

Larsson P G, Carlsson B, Fahraeus L, Jakobsson T, Forsum U. Diagnosis of bacterial vaginosis: need for validation of microscopic image area used for scoring bacterial morphotypes. Sex Transm Infect. 2004;80:63-7.

Leitich H, Brunbauer M, Bodner-Adler B, Kaider A, Egarter C, Husslein P. Antibiotic treatment of bacterial vaginosis in pregnancy: a meta-analysis. Am J Obstet Gynecol. 2003;188:752-8.

Libman M D, Kramer M, Platt R; Montreal Prematurity Study Group. Comparison of Gram and Kopeloff stains in the diagnosis of bacterial vaginosis in pregnancy. Diagn Microbiol Infect Dis. 2006;54:197-201.

McDonald H, Brocklehurst P, Parsons J. Antibiotics for treating bacterial vaginosis in pregnancy. Cochrane Database Syst Rev. 2005 Jan. 25; (1):CD000262. Review.

Morales H J, Schorr S and J Albritton, Effect of metronidazole in patients with preterm birth in preceding pregnancy and bacterial vaginosis: a placebo-controlled, double-blind study, Am J Obstet Gynecol 171 (1994), pp. 345-347.

Nugent R P, Krohn M A, Hillier S L. Reliability of diagnosing bacterial vaginosis is improved by a standardized method of gram stain interpretation. J Clin Microbiol. 1991;29:297-301.

Okun N, Gronau K A, Hannah M E. Antibiotics for bacterial vaginosis or Trichomonas vaginalis in pregnancy: a systematic review. Obstet Gynecol. 2005;105:857-68.

Rodriguez Jovita M, Collins M D, Sjoden B, Falsen E. Characterization of a novel Atopobium isolate from the human vagina: description of Atopobium vaginae sp. nov. Int J Syst Bacteriol. 1999;49:1573-6.

Schwebke J R, Hillier S L, Sobel J D, McGregor J A, Sweet R L. Validity of the vaginal gram stain for the diagnosis of bacterial vaginosis. Obstet Gynecol. 1996;88:573-6.

Schwebke J R, Lawing L F. Prevalence of Mobiluncus spp. among women with and without bacterial vaginosis as detected by polymerase chain reaction. Sex Transm Dis. 2001;28:195-9.

Secor, A. M. 1997. Diagnosis and treatment of chronic vulvovaginitis. Clin Nurse Practitioner 1:1-6.

Sha B E, Chen H Y, Wang Q J, Zariffard M R, Cohen M H, Spear G T. Utility of Amsel criteria, Nugent score, and quantitative PCR for Gardnerella vaginalis, Mycoplasma hominis, and Lactobacillus sp. for diagnosis of bacterial vaginosis in human immunodeficiency virus-infected women. J Clin Microbiol. 2005;43:4607-12

Spiegel C A., Bacterial Vaginosis. Clinical Microbiology Reviews. 1991; 4:485-502.

Thomason J L, Anderson R J, Gelbart S M, Osypowski P J, Scaglione N J, el Tabbakh G, James J A. Simplified gram stain interpretive method for diagnosis of bacterial vaginosis. Am J Obstet Gynecol. 1992;167:16-9.

Thorsen P, Jensen I P, Jeune B, Ebbesen N, Arpi M, Bremmelgaard A, Moller B R. Few microorganisms associated with bacterial vaginosis may constitute the pathologic core: a population-based microbiologic study among 3596 pregnant women. Am J Obstet Gynecol. 1998;178:580-7.

Ugwumadu A, Manyonda I, Reid F, Hay P. Effect of early oral clindamycin on late miscarriage and preterm delivery in asymptomatic women with abnormal vaginal flora and bacterial vaginosis: a randomised controlled trial. Lancet. 2003;361:983-8.

Varma R, Gupta J K. Antibiotic treatment of bacterial vaginosis in pregnancy: multiple meta-analyses and dilemmas in interpretation. Eur J Obstet Gynecol Reprod Biol. 2006;124:10-4.

Verhelst, R., H. Verstraelen, G. Claeys, G. Verschraegen, J. Delanghe, L. Van Simaey, C. De Ganck, M. Temmerman, and M. Vaneechoutte. 2004. Cloning of 16S rRNA genes amplified from normal and disturbed vaginal microflora suggests a strong association between Atopobium vaginae, Gardnerella vaginalis and bacterial vaginosis. BMC. Microbiol 2004; 4:16.

Verhelst R, Verstraelen H, Claeys G, Verschraegen G, Van Simaey L, De Ganck C, De Backer E, Temmerman M, Vaneechoutte M. Comparison between Gram stain and culture for the characterization of vaginal microflora: definition of a distinct grade that resembles grade I microflora and revised categorization of grade I microflora. BMC Microbiol. 2005 Oct. 14; 5:61.

Zariffard M R, Saifuddin M, Sha B E, Spear G T. Detection of bacterial vaginosis-related organisms by real-time PCR for Lactobacilli, Gardnerella vaginalis and Mycoplasma hominis. FEMS Immunol Med Microbiol. 2002 13;34:277-81.

TABLE 1
Bacterial culture results for the three flora types identified by the Nugent score.

| | Culture | | | |
|---|---|---|---|---|
| | | Intermediate | Bacterial | |
| | Normal Flora | Flora | Vaginosis | Total |
| | Number of positive specimens as percentage of | | | |
| Microorganism to be detected | number of tested specimens | | | |
| G. vaginalis | 6 out of 167 | 6 out of 44 | 15 out of 20 | 27 out of 231 |
| | (3.6%) | (13.6%) | (75%) | (12%) |
| M. hominis and U. urealyticum | 16 out of 163 | 6 out of 39 | 4 out of 16 | 26 out of 2 |
| | (9.8%) | (15%) | (25%) | (9%) |
| C. albicans | 30 out of 167 | 17 out of 44 | 3 out of 20 | 50 out of 231 |
| | (18%) | (38.6%) | (15%) | (21.6%) |
| Streptococcus agalactiae | 5 out of 167 | 1 out of 44 | 0 out of 20 | 6 out of 231 |
| | (3%) | (2%) | | (2.6%) |

TABLE 2
Detection of the presence of 8 microorganisms by real-time
PCR for the 231 specimens

| Microorganisms | Number and percentage of positive PCRs |
|---|---|
| targeted by PCR | (positivity threshold 10³/mL) |
| Lactobacillus sp. | 177 (77%) |
| G. vaginalis | 131 (57%) |
| A. vaginae | 172 (74%) |
| M. mulieris | 1 (0.4%) |
| M. curtisii | 41 (18%) |
| M. hominis | 47 (20%) |
| U. urealyticum | 32 (14%) |
| C. albicans | 60 (26%) |

TABLE 3
Analysis of median bacterial concentrations obtained by quantitative PCR for
bacterial vaginosis and normal vaginal flora determined by the Nugent score.

| | 20 bacterial vaginoses | 167 normal flora | P value |
|---|---|---|---|
| | Median bacterial concentration/mL (minimum and | | Mann-Whitney |
| Microorganisms | maximum concentration) | | statistical test |
| Lactobacillus sp. | 2.7 × 10³ (<10³-8 × 10⁷) | 5.6 × 10⁷ (<10³-5.4 × 10⁹) | <0.0001 |
| G. vaginalis | 1.1 × 10⁹ (<10³-1.5 × 10¹⁰) | <10³ (<10³-5 × 10⁸) | <0.0001 |
| A. vaginae | 1.2 × 10⁹ (<10³-1.2 × 10¹⁰) | 4.7 × 10³ (<10³-2 × 10⁸) | <0.0001 |
| M. mulieris | <10³ | <10³ | not measurable |
| M. curtisii | 4.9 × 10³ (<10³-1.3 × 10⁶) | <10³ (<10³-6.2 × 10⁴) | <0.0001 |
| M. hominis | 8.5 × 10² (<10³-1.3 × 10⁸) | <10³ (<10³-2.2 × 10⁹) | 0.0054 |
| U. urealyticum | <10³ (<10³-3.4 × 10⁵) | <10³ (<10³-5 × 10⁶) | 0.3344 |
| C. albicans | <10³ (<10³-1.7 × 10³) | <10³ (<10³-7 × 10⁶) | 0.0991 |

TABLE 4
Impact of bacterial quantification by real-time PCT for identification of
bacterial vaginosis.
Group of normal flora and bacterial vaginoses defined by the Nugent
score used as reference.

| | Quantification | | | | | | | | |
|---|---|---|---|---|---|---|---|---|---|
| | threshold | | | | | | | | |
| Microorganisms | (bacteria/mL) | NF identified | | BV identified* | | Sensitivity** | Specificity** | PPV*** | NPV*** |
| A. vaginae | ≧10³ | 116 | (69%) | 19 | (95%) | 0.95 | 0.31 | 0.14 | 0.98 |
| | ≧10⁴ | 65 | (39%) | 19 | (95%) | 0.95 | 0.61 | 0.23 | 0.99 |
| | ≧10⁵ | 25 | (15%) | 19 | (95%) | 0.95 | 0.85 | 0.43 | 0.99 |
| | ≧10⁶ | 13 | (7.8%) | 19 | (95%) | 0.95 | 0.92 | 0.59 | 0.99 |
| | ≧10⁷ | 6 | (3.6%) | 19 | (95%) | 0.95 | 0.96 | 0.76 | 0.99 |
| | ≧10⁸ | 1 | (0.6%) | 18 | (90%) | 0.90 | 0.99 | 0.95 | 0.99 |
| | ≧10⁹ | 0 | | 13 | (65%) | 0.65 | 1.00 | 1.00 | 0.96 |
| G. vaginalis | ≧10³ | 79 | (47%) | 19 | (95%) | 0.95 | 0.53 | 0.19 | 0.99 |
| | ≧10⁴ | 59 | (35%) | 19 | (95%) | 0.95 | 0.65 | 0.24 | 0.99 |
| | ≧10⁵ | 39 | (23%) | 19 | (95%) | 0.95 | 0.77 | 0.33 | 0.99 |
| | ≧10⁶ | 25 | (15%) | 19 | (95%) | 0.95 | 0.85 | 0.43 | 0.99 |
| | ≧10⁷ | 12 | (7%) | 18 | (90%) | 0.90 | 0.93 | 0.60 | 0.99 |
| | ≧10⁸ | 7 | (4%) | 16 | (80%) | 0.80 | 0.96 | 0.70 | 0.98 |
| | ≧10⁹ | 0 | | 10 | (50%) | 0.50 | 1.00 | 1.00 | 0.94 |
| M. curtisii | ≧10³ | 21 | (12.6%) | 13 | (65%) | 0.65 | 0.87 | 0.38 | 0.95 |
| | ≧10⁴ | 2 | (1.2%) | 9 | (45%) | 0.45 | 0.99 | 0.82 | 0.94 |
| | ≧10⁵ | 0 | | 9 | (45%) | 0.45 | 1.00 | 1.00 | 0.94 |
| | ≧10⁶ | 0 | | 1 | (5%) | 0.05 | 1.00 | 1.00 | 0.90 |
| M. hominis | ≧10³ | 22 | (13%) | 10 | (50%) | 0.50 | 0.87 | 0.31 | 0.94 |
| | ≧10⁴ | 13 | (7.8%) | 7 | (35%) | 0.35 | 0.92 | 0.35 | 0.92 |
| | ≧10⁵ | 6 | (3.6%) | 7 | (35%) | 0.35 | 0.96 | 0.54 | 0.93 |
| | ≧10⁶ | 2 | (1.2%) | 6 | (30%) | 0.30 | 0.99 | 0.75 | 0.92 |
| | ≧10⁷ | 2 | (1.2%) | 6 | (30%) | 0.30 | 0.99 | 0.75 | 0.92 |
| | ≧10⁸ | 2 | (1.2%) | 1 | (5%) | 0.05 | 0.99 | 0.33 | 0.90 |
| | ≧10⁹ | 1 | (0.6%) | 0 | | 0 | 0.99 | 0 | 0.89 |
| U. urealyticum | ≧10³ | 22 | (13%) | 5 | (25%) | 0.25 | 0.87 | 0.18 | 0.91 |
| | ≧10⁴ | 17 | (10%) | 5 | (25%) | 0.25 | 0.90 | 0.23 | 0.91 |
| | ≧10⁵ | 13 | (7.8%) | 3 | (15%) | 0.15 | 0.92 | 0.19 | 0.90 |
| | ≧10⁶ | 7 | (4.2%) | 0 | | 0 | 0.96 | 0 | 0.89 |
| Lactobacillus | ≧10³ | 141 | (84%) | 12 | (60%) | 0.60 | 0.16 | 0.08 | 0.76 |
| spp. | ≧10⁴ | 134 | (80%) | 7 | (35%) | 0.35 | 0.20 | 0.05 | 0.72 |
| | ≧10⁵ | 116 | (69%) | 4 | (20%) | 0.20 | 0.30 | 0.03 | 0.76 |
| | ≧10⁶ | 106 | (63%) | 4 | (20%) | 0.20 | 0.36 | 0.04 | 0.79 |
| | ≧10⁷ | 97 | (58%) | 3 | (15%) | 0.15 | 0.42 | 0.03 | 0.80 |
| | ≧10⁸ | 74 | (44%) | 0 | | 0 | 0.55 | 0 | 1 |
| | ≧10⁹ | 13 | (7.8%) | 0 | | 0 | 0.92 | 0 | 1 |

*No of NF and no. of BV identified out of 167 NF tested (%) and 20 BV tested (%), respectively.
**Sensitivity and specificity of quantification threshold for BV identification
***PPV: positive predictive value; NPV: negative predictive value

TABLE 5
Impact of quantification criteria by real-time PCR of A. vaginae and G. vaginalis
for identification of bacterial vaginosis. Group of normal flora and bacterial
vaginoses defined by Nugent score used as reference.

| | G. vaginalis | | | | | | | | |
|---|---|---|---|---|---|---|---|---|---|
| | quantification | | | | | | | | |
| Associated | threshold | NF | | | | | | | |
| microorganisms | (bacteria/mL) | identified* | | BV identified* | | Sensitivity** [symbol] | Specificity** | PPV** | NPV** |
| A. vaginae ≧ 10⁷ and | ≧10⁶ | 5 | (3%) | 18 | (90%) | 0.90 | 0.97 | 0.78 | 0.99 |
| G. vaginalis | ≧10⁷ | 5 | (3%) | 17 | (85%) | 0.85 | 0.97 | 0.77 | 0.98 |
| | ≧10⁸ | 2 | (1.2%) | 15 | (75%) | 0.75 | 0.99 | 0.88 | 0.97 |
| | ≧10⁹ | 0 | | 9 | (45%) | 0.45 | 1 | 1 | 0.94 |
| A. vaginae ≧ 10⁸ and | ≧10⁶ | 1 | (0.6%) | 17 | (85%) | 0.85 | 0.99 | 0.94 | 0.98 |
| G. vaginalis | ≧10⁷ | 1 | (0.6%) | 17 | (85%) | 0.85 | 0.99 | 0.94 | 0.98 |
| | ≧10⁸ | 0 | | 15 | (75%) | 0.75 | 1 | 1 | 0.97 |
| | ≧10⁹ | 0 | | 9 | (45%) | 0.45 | 1 | 1 | 0.94 |
| A. vaginae ≧ 10⁷ | ≧10⁶ | 26 | (15%) | 20 | (100%) | 1 | 0.84 | 0.43 | 1 |
| and/or G. vaginalis | ≧10⁷ | 13 | (7.8%) | 20 | (100%) | 1 | 0.92 | 0.61 | 1 |
| | ≧10⁸ | 11 | (6.6%) | 20 | (100%) | 1 | 0.93 | 0.64 | 1 |
| | ≧10⁹ | 6 | (3.6%) | 20 | (100%) | 1 | 0.96 | 0.77 | 1 |
| A. vaginae ≧ 10⁸ | ≧10⁶ | 25 | (15%) | 20 | (100%) | 1 | 0.85 | 0.44 | 1 |
| and/or G. vaginalis | ≧10⁷ | 12 | (7.2%) | 19 | (95%) | 0.95 | 0.93 | 0.61 | 0.99 |
| | ≧10⁸ | 7 | (4.2%) | 19 | (95%) | 0.95 | 0.96 | 0.73 | 0.99 |
| | ≧10⁹ | 1 | | 19 | (95%) | 0.95 | 0.99 | 0.95 | 0.99 |

*No. of NF and no. of BV identified out of 167 NF tested (%) and 20 BV tested (%), respectively.
**Sensitivity and specificity of quantification threshold for BV identification
***PPV: positive predictive value; NPV: negative predictive value

TABLE 6
Bacteriology follow-ups of 8 patients with bacterial vaginosis or an
intermediate flora by Nugent score. Correspondence between classification
by Nugent score and BV identification by molecular criteria.

| | | Classification | | | | |
|---|---|---|---|---|---|---|
| | Time vaginal | par Score of | | BV by molecular | Length of pregnancy | Newborn |
| Subject | specimen taken | Nugent | Treatment | criteria | at delivery | weight (grams) |
| 1 | 24 WA | BV | Yes | yes | 39 WA | 3420 |
| | 28 WA | NF | | | | |
| | 38 WA | NF | | yes | | |
| 2 | 27 WA | IF | | yes | 40 WA | 3090 |
| | 39 WA | IF | | yes | | |
| 3 | 6 WA | NF | | | 40 WA | 3490 |
| | 28 WA | IF | | | | |
| | 32 WA | IF | | | | |
| 4 | 6 WA | IF | | yes | 38 WA | 3580 |
| | 31 WA | NF | | | | |
| | 37 WA | NF | | | | |
| | 40 WA | NF | | | | |
| 5 | 26 WA | IF | | yes | 38 WA | 2970 |
| | 31 WA | IF | | yes | | |
| 6 | 31 WA | NF | | | 39 WA | 2700 |
| | 39 WA | IF | | yes | | |
| 7 | 25 WA | BV | Yes | yes | 37 WA | 4100 |
| | 29 WA | NF | | | | |
| 8 | 27 WA | BV | Yes | yes | 38 WA | 2700 |
| | 30 WA | NF | | | | |
| | 32 SA | NF | | | | |

1. A method for in vitro diagnosis and follow-up of the vaginal bacterial flora status in relation to the presence of bacterial vaginosis, and for monitoring its treatment where applicable, the method comprising:
1) quantifying the concentrations of the bacteria Atopobium vaginae and Gardnerella vaginalis by
determining the concentrations of-the specific sequences of said bacteria Atopobium vaginae and Gardnerella vaginalis present in a single copy in the DNA of said bacteria Atopobium vaginae and Gardnerella vaginalis, and of a specific sequence of a human gene present in all biological specimens containing human cells, in the DNA extracted from a vaginal discharge specimen from a patient, said specific sequences being less than 150 nucleotides in size,
enzymatically co-amplifying said specific sequences contained, on the one hand, in said DNA extracted from the specimen and, on the other hand, in samples of synthetic DNA fragments including each of said specific sequences of said bacteria and said specific sequence of a human gene present in all biological human cell specimens, said samples serving as calibration standards for quantifying the DNA,
detecting and quantifying said amplified fragments with the aid of probes labeled with sequences different from those of the amplification primers for each of said specific sequences of said bacteria Atopobium vaginae and Gardnerella vaginalis and said specific sequence of a human gene present in all biological human cell specimens, and
2) determining the presence of bacterial vaginosis or failure of the on-going treatment if the concentrations of DNA fragments of said two specific sequences of the bacteria Atopobium vaginae and Gardnerella vaginalis in a specimen of the patient's vaginal discharge containing at least 104 human cells/mL are such that at least one of the following two conditions a) and b) is met:
a) the concentration Ca of said DNA fragment of Atopobium vaginae is greater than or equal to 108 copies/mL, and
b) the concentration Cg of said DNA fragment of Gardnerella vaginalis is greater than or equal to 109 copies/mL.

2. The method according to claim 1, wherein:
a) in step 1), the Lactobacillus sp. including at least the bacteria Lactobacillus acidophilus, Lactobacillus crispatus, Lactobacillus jenseneii, Lactobacillus gasseri and Lactobacillus iners are additionally quantified
by additionally determining the concentration of a specific sequence of said Lactobacillus sp. bacteria, said specific sequence of Lactobacillus sp. being present in a single copy in the DNA of said Lactobacillus sp. bacteria, and being less than 150 nucleotides in size,
by additionally ezymatically co-amplifying said specific sequence of Lactobacillus sp. contained, on the one hand, in said DNA extracted from the specimen and, on the other hand, in a specimen of synthetic DNA fragments including, additionally, said specific sequence of said Lactobacillus sp. bacteria, with said synthetic DNA fragment including said specific sequence of said Lactobacillus sp. bacteria serving as a quantification reference standard.
detection and quantification of said amplified fragments being accomplished with the aid of labeled probes with sequences different from those of the amplification primers for each of said specific sequences of said Atopobium vaginae, Gardnerella vaginalis, and Lactobacillus sp. bacteria and said specific sequence of a human gene present in any biological specimen containing human cells, and
b) in step 2), bacterial vaginosis is determined if, additionally, the Cl concentration of a specific DNA fragment of said Lactobacillus sp. bacteria is less than or equal to 108 copies/mL.

3. The method according to claim 2, wherein the presence of bacterial vaginosis or failure of the on-going treatment is determined if said concentrations are such that the following three conditions are met:
a—Concentration Ca of said DNA fragment of a specific sequence of Atopobium vaginae is greater than or equal to 108 copies/mL,
b—Concentration Cg of said DNA fragment of a specific sequence of Gardnerella vaginalis is greater than or equal to 109 copies/mL, and
c—Concentration Cl of said DNA fragment of a specific sequence of Lactobacillus sp. is less than or equal to 107 copies/mL.

4. The method according to claim 1, wherein real-time PCR amplification and quantification reactions are performed by using hydrolysis probes specific to each of said specific sequences of said bacteria and specific sequence of a human gene present in any biological specimen containing human cells, in the specimen to be tested.

5. The method according to claim 4, wherein said specific sequences are 70 to 150 nucleotides in size.

6. The method according to claim 1, wherein a large synthetic DNA fragment serving as a reference standard for DNA quantification is used, said large synthetic DNA fragment grouping said specific sequences of each of said bacteria whose concentrations are quantified, and said specific human DNA sequence of human cells.

7. The method according to claim 1, wherein said specific sequences of said bacteria include:
for the Atopobium vaginae bacterium: the fragment of positions 248 to 334 of the 16S ribosomal RNA gene, GenBank reference AY 738658.1,
for the Gardnerella vaginalis bacterium, the fragment of positions 981 to 1072 of the Cpn 60 gene of the chaperone protein 60 kDa, GenBank reference AF 240579.3, and
for the Lactobacillus sp. bacterium, a sequence common to the bacteria Lactobacillus crispatus, Lactobacillus jenseneii, Lactobacillus gasseri, Lactobacillus iners and Lactobacillus acidophilus in the tuf gene coding for the elongation factor at positions 253 to 343 of the gene with GenBank reference AY 562191.1.

8. The method according to claim 7, wherein said specific sequences of said bacteria are the following sequences, including probe sequences framed by primer sequences or their complementary sequences:
the sequence as set forth in SEQ ID NO:2 for Atopobium vaginae,
the sequence as set forth in SEQ ID NO:3 for Gardnerella vaginalis, and
the sequence as set forth in SEQ ID NO:4 for Lactobacillus sp.

9. The method according to claim 6, wherein said large DNA fragment includes, as a specific sequence of human DNA in the test sample, a specific albumin sequence.

10. The method according to claim 9, wherein said specific sequence of human DNA in the test specimen includes the fragment of positions 16283-16423 of exon 12 of the human albumin gene with GenBank reference M12523.1 with the sequence as set forth in SEQ ID NO:1 or the complementary sequence thereof.

11. The method according to claim 10, wherein said specific sequence of the human DNA in the test specimen including the fragment of positions 16283-16423 of exon 12 of the human albumin gene GenBank reference M12523.1, is modified by insertion of a cleavage site outside the sequences corresponding to the primers and the probe sequence of the sequence as set forth in SEQ ID NO:17.

12. The method according to claim 1, wherein sets of primers and probes are chosen where applicable from the following sequences or their complementary sequences:
for Atopobium vaginae
SEQ ID NOs:5-7 for the 5′ primer, the 3′ primer, and the probe, respectively;
for Gardnerella vaginalis
SEQ ID NOs:8-10 for the 5′ primer, the 3′ primer, and the probe, respectively;
for Lactobacillus sp.
SEQ ID NOs:11-13 for the 5′ primer, the 3′ primer, and the probe, respectively; and
for human albumin
SEQ ID NOs:14-16 for the 5′ primer, the 3′ primer, and the probe, respectively.

13. The method according to claim 6, wherein said large synthetic DNA fragment constituting the DNA of the standard reference specimen for quantification is inserted into a plasmid.

14. Diagnostic kit used for implementing a method according to claim 1 comprising:
samples of standard reference DNA including said specific sequences of each of said bacteria, as well as
said sets of specific primers of said modified synthetic DNA fragments specific to said bacteria, and
reagents for implementing a PCR-type DNA amplification reaction.


SEQUENCE LISTING (28 sequences)

SEQ ID NO: 1 - DNA, 135 bp, artificial sequence
  Sequence of human albumin
gctgtcatctcttgtgggctgtaatcatcgtttaagagtaatattgcaaaacctgtcatgcccacacaaatctctccctggcattgttgtctttgcagatgtcagtgaaagagaaccagcagctcccatgagttt

SEQ ID NO: 2 - DNA, 81 bp, Atopobium vaginae
ccctatccgctcctgataccggcaggcttgagtctggtaggggaagatggaattccaagtgtgaaatgcgcagatatttgg

SEQ ID NO: 3 - DNA, 86 bp, Gardnerella vaginalis
cgcatctgctaaggatgttgaaacatcgtttaaggctactgcaactatttctgcagcagatcctgaagttggcgaaaagattgctg

SEQ ID NO: 4 - DNA, 90 bp, Lactobacillus sp.
tacatcccaactccagaacgtgatactgacaagccattcttaatgcagttgaagacgtatttactatcactggtcgtggtactgttgctt

SEQ ID NO: 5 - DNA, 20 bp, Atopobium vaginae
ccctatccgctcctgatacc

SEQ ID NO: 6 - DNA, 20 bp, Atopobium vaginae
ccaaatatctgcgcatttca

SEQ ID NO: 7 - DNA, 23 bp, Atopobium vaginae
gcaggcttgagtctggtagggga

SEQ ID NO: 8 - DNA, 20 bp, Gardnerella vaginalis
cgcatctgctaaggatgttg

SEQ ID NO: 9 - DNA, 20 bp, Gardnerella vaginalis
cagcaatcttttcgccaact

SEQ ID NO: 10 - DNA, 24 bp, Gardnerella vaginalis
tgcaactatttctgcagcagatcc

SEQ ID NO: 11 - DNA, 20 bp, Lactobacillus sp.
tacatcccaactccagaacg

SEQ ID NO: 12 - DNA, 20 bp, Lactobacillus sp.
aagcaacagtaccacgacca

SEQ ID NO: 13 - DNA, 21 bp, Lactobacillus sp.
tgacaagccattcttaatgca

SEQ ID NO: 14 - DNA, 22 bp, artificial sequence
  Sequence of human albumin
gctgtcatctcttgtgggctgt

SEQ ID NO: 15 - DNA, 23 bp, artificial sequence
  Sequence of human albumin
aaactcatgggagctgctggttc

SEQ ID NO: 16 - DNA, 26 bp, artificial sequence
  Sequence of human albumin
cctgtcatgcccacacaaatctctcc

SEQ ID NO: 17 - DNA, 141 bp, artificial sequence
  Fragment of exon 12 of gene human albumin of positions 16283 to 16423 as modified by inserting the cleavage site XhoI
gctgtcatctcttgtgggctgtaatcatcgtctcgagttaagagtaatattgcaaaacctgtcatgcccacacaaatctctccctggcattgttgtctttgcagatgtcagtgaaagagaaccagcagctcccatgagttt

SEQ ID NO: 18 - DNA, 939 bp, artificial sequence
  Sequence combining sequences of Mobilincus curtisii, Mobilincus mulieris, Per vaginalis, Lactobacillus sp, HIV patients, Mycoplasma hominis, Atopobium vaginae, Candida albicans and human albumin
gccatggaaaaggtgggtcaagagggcgtcatcaccgtggaagaacatcgtctcgagttaaggagcctcgaagtcaccgaaggtatgcgtttcattatggatatgcgtgtggatggattactcgagctgcctgttttgggtgggggcgctatcggggtttgggcttacatgcctggccctcgcatctgctaaggatgttgaaacatcgtctcgagttaaggctactgcaactatttctgcagcagatcctgaagttggcgaaaagattgctgaatacatcccaactccagaacgtgatactgacaagccattcttaatgccagttgaagacgtatttactatcactggtcgtggtactgttgcttctatactggtgaccgtcctatccaagttggatcacatcgtctcgagttaagaacaaatagtgcattagtattctttgatgaaaaaggaaacgaagacaaagaacgtaaagttgcttatggacgtcgtttcgatattccatcaggtaattgattgctgcaggtgatacatttagagcagcggcagttgaacaattcatcgtctcgagttaaggagttggggctgatattgtaacaccaaacccctatccgctcctgataccggcaggcttgagtctggtaggggaagatggaattccaagtctcgaggtgaaatgcgcagatatttggaagccaacgccaacgaagacaaggcacttcaaaaagccgatggtagtagaaaactgcgacatcgtctcgagttaagttggttgatgcaaacaaagctggtagttgctgtcatctcttgtgggctgtaatcatcgtctcgagttaagagtaatattgcaaaacctgtcatgcccacacaaatctctccctggcattgttgtctttgcagatgtcagtgaaagagaaccagcagctcccatgagtttgg

SEQ ID NO: 19 - DNA, 178 bp, artificial sequence
  Sequence of Mobilincus curtisii and Mobilincus mulieris
gccatggaaaaggtgggtcaagagggcgtcatcaccgtggaagaacatcgtctcgagttaaggagcctcgaagtcaccgaaggtatgcgtttcattatggatatgcgtgtggatggattactcgagctgcctgttttgggtgggggcgctatcggggtttgggcttacatgcctggcc

SEQ ID NO: 20 - DNA, 183 bp, artificial sequence
  Sequence of Per vaginalis and Lactobacillus sp
cgaccagtgatagtaaatacgtcttcaactggcattaagaatggcttgtcagtatcacgttctggagttgggatgtattcagcaatcttttcgccaacttcaggatctgctgcagaaatagttgcagtagccttaactcgagacgatgtttcaacatccttagcagatgcgagggccaggcat

SEQ ID NO: 21 - DNA, 145 bp, Ureaplasma urealyticum
tcactggtcgtggtactgttgcttctatactggtgaccgtcctatccaagttggatcacatcgtctcgagttaagaacaaatagtgcattagtattctttgatgaaaaaggaaacgaagacaaagaacgtaaagttgcttatgga

SEQ ID NO: 22 - DNA, 188 bp, Mycoplasma hominis
cttggaattccatcttcccctaccagactcaagcctgccggtatcaggagcggataggggtttggtgttacaatatcagccccaactccttaactcgagacgatgaattgttcaactgccgctgctctaaatgtatcacctgcagcaatcaattacctgatggaatatcgaaacgacgtccataagca

SEQ ID NO: 23 - DNA, 150 bp, artificial sequence
  Sequence of Atopobium vaginae and Candida albicans
gaattccaagtctcgaggtgaaatgcgcagatatttggaagccaacgccaacgaagacaaggcacttcaaaaagccgatggtagtagaaaactgcgacatcgtctcgagttaagttggttgatgcaaacaaagctggtagttgctgtcat

SEQ ID NO: 24 - DNA, 145 bp, artificial sequence
  Sequence of human albumin
ccaaactcatgggagctgctggttctctttcactgacatctgcaaagacaacaatgccagggagagatttgtgtgggcatgacaggttttgcaatattactcttaactcgagacgatgattacagcccacaagagatgacagcaa

SEQ ID NO: 25 - DNA, 19 bp, artificial sequence
  Synthetic construct
gccatggaaaaggtgggtc

SEQ ID NO: 26 - DNA, 21 bp, artificial sequence
  Synthetic construct
cctgatggaatatcgaaacga

SEQ ID NO: 27 - DNA, 20 bp, artificial sequence
  Synthetic construct
actggtgaccgtcctatcca

SEQ ID NO: 28 - DNA, 20 bp, artificial sequence
  Synthetic construct
ccaaactcatgggagctgct